(19) United States
(12) Patent Application Publication
(10) Pub. No.: US 2024/0328957 A1
(43) Pub. Date: Oct. 3, 2024

(54) CHEMILUMINESCENCE MICROFLUIDIC IMMUNOASSAY DEVICE AND METHODS OF USE THEREOF

(71) Applicants: Burst Diagnostics LLC, Fort Collins, CO (US); Zymo Research Corporation, Irvine, CA (US)

(72) Inventors: Zach Call, Fort Collins, CO (US); Rose Nash, Fort Collins, CO (US); Alli Dolence, Fort Collins, CO (US); Charles Henry, Fort Collins, CO (US); Joseph Ollar, Newport Beach, CA (US); Xi Yu Jia, Newport Beach, CA (US)

(21) Appl. No.: 18/625,514

(22) Filed: Apr. 3, 2024

Related U.S. Application Data
(60) Provisional application No. 63/456,655, filed on Apr. 3, 2023.

(51) Int. Cl. G01N 21/76; G01N 33/58

(52) U.S. Cl. CPC G01N 21/76; G01N 33/581

(57) ABSTRACT

A microfluidic assay device having a chemiluminescent testing assembly including a detection area, a sample inlet, and a microfluidic network including a first and second path extending to the detection area configured to capture a target analyte. When a fluid is provided to the sample inlet, a first portion of the fluid rehydrates a first dried reagent disposed along the first path to produce a first rehydrated reagent and a second portion of the fluid rehydrates a second dried reagent to produce a second rehydrated reagent. The first rehydrated reagent and the second rehydrated reagent are then sequentially delivered to the detection area by capillary-driven flow to perform the assay, where they react, optionally in the presence of a catalyst to form a chemiluminescent signal.

### CROSS-REFERENCE TO RELATED APPLICATIONS

This U.S. Non-Provisional application claims the benefit of and priority to U.S. Provisional Application No. 63/456,655 filed Apr. 3, 2023, the specification, claims and drawings of which are incorporated herein by reference in their entirety.

### TECHNICAL FIELD

The present invention is directed to the field of microfluidic diagnostic devices, and specifically chemiluminescent microfluidic diagnostic devices and their methods of use to detect and quantify target analytes in fluid samples.

### BACKGROUND

The targeted detection of analytes from biological and environmental samples has become the cornerstone of many diagnostic and therapeutic processes. While analytical methods like PCR and ELISA exist for detection of analytes, such as biomarkers and other compounds, they remain time consuming and expensive. Lateral flow assays (LFAs) are an attractive option for point-of-care testing. However, these devices are limited by spatial resolution of test lines, large sample volumes, cross-reactivity, and poor sensitivity. As described by Henry et al., in PCT/US2021/071050—incorporated herein by reference, colorimetric capillary-flow microfluidic ELISA platform are a more sensitive alternative to LFAs. However, there exist a need for microfluidic assays that exhibit even higher sensitivities that traditional colorimetric point-of-care immunoassay systems without significantly increasing cost.

To overcome these limitations, the present inventors describe herein an inexpensive and highly sensitive chemiluminescent capillary-flow immunoassay device configured to detect target analytes from fluid samples, including biological and environmental samples. As further described herein, chemiluminescent results of the device can be imaged and processed by a computer executable program, for example using a smartphone or other imaging device, to generate additional quantitative data.

### SUMMARY OF THE INVENTION

In one aspect, the present invention includes systems, methods and devices directed to a novel chemiluminescent assay. In this preferred aspect, the chemiluminescent assay device of the invention includes a chemiluminescent testing assembly further including a sample inlet for receiving a fluid sample in communication with a microfluidic network formed by a first and a second path extending to a detection area, wherein that detection area is configured to capture a target analyte, for example by a capture probe configured to bind to a target analyte in a fluid sample disposed of on the detection area. In this preferred aspect, a first dried reagent is disposed along the first path, while a second dried reagent disposed along the second path. In this configuration, when a fluid is provided to the sample inlet the fluid fills the microfluidic network and directs a portion of the fluid through the microfluidic channel to the detection area where the target analyte, if present in the sample, is captured, and wherein a first portion of the fluid rehydrates the first dried reagent to produce a first rehydrated reagent, a second portion of the fluid rehydrates the second dried reagent to produce a second rehydrated reagent, and the first rehydrated reagent and the second rehydrated reagent are sequentially delivered to the detection area by capillary-driven flow, and wherein the first reagent, and the second reagent react in the presence of a catalyst, the reaction emitting an chemiluminescent signal. The sample inlet may include a filtration membrane in certain aspects.

In another aspects of the invention, the chemiluminescent assay device of the invention includes a pad. In a preferred aspect, the pad may be disposed within the first path and contain the first dried reagent, or wherein the pad can be disposed within the second path and contains the second dried reagent.

In another aspects of the invention, the first path includes a first surface on which a first dried reagent is disposed, or the second path includes a second surface on which a second dried reagent is disposed. In this preferred aspect, the microfluidic network is configured such that the fluid arrives at the detection area by the first path before the fluid arrives at the detection area by the second path. In this preferred aspect, the first path is shorter than the second path, that that in this configuration the fluid, carrying the first rehydrated reagent arrives at the detection area before the fluid containing the first rehydrated reagent.

In another aspects of the invention, the chemiluminescent assay device of the invention includes a body formed from alternating layers of film and double-sided adhesive, wherein the microfluidic network is formed by the layers of film and double-sided adhesive. In another preferred aspect, the chemiluminescent assay device of the invention includes one or more vents in communication with the microfluidic network.

In another aspect of the invention, the chemiluminescent assay device of the invention includes a second colorimetric testing assembly including a second detection area and a second microfluidic network in communication with each of the fluid inlet and the second detection area.

In another aspect of the invention, the chemiluminescent assay device of the invention, the first rehydrated reagent comprises an enzyme label rehydrated from a dried enzyme label pad. In a preferred aspect, the enzyme label comprises a secondary antibody directed to a target analyte.

In another aspect of the invention, the chemiluminescent assay device of the invention, the second rehydrated reagent comprises an a substrate rehydrated from a dried substrate pad. In a preferred aspect, the substrate comprises a chemiluminescent substrate configured to react with the enzyme label and emit chemiluminescent signal, such as preferably the emission of a quantity of photons, in the presence of a catalyst. In this one preferred aspect, the chemiluminescent substrate can include, but not be limited to: luminol, luminol analogues, adamantyl 1, 2-dioxetane aryl phosphate (AMPPD), 4-methylumbelliferyl phosphate (4-MUP), Luciferin, a substrate of alkaline phosphatase (ALP), a substrate of horseradish peroxidase (HPR), a substrate of Poly-horseradish peroxidase (HPR), and a substrate of luciferase, or a combination of the same.

In another aspect of the invention, the chemiluminescent assay device of the invention, the catalyst can include peroxide or other oxidizing agent. In a preferred aspect, the catalyst, in this aspect being peroxide or other oxidizing agent is disposed of within the fluid, or within a buffer, or on the dried substrate pad, or on the test strip.

In another aspect of the invention, the chemiluminescent assay device of the invention, the fluid comprises a biological sample, or an environmental sample, or a food or water sample. In a preferred aspect, the fluid comprises a biological sample, or an environmental sample or a food or water sample, preferably diluted in a buffer.

In another aspect of the invention, the chemiluminescent assay device of the invention, the detection area comprises a test strip, such as a LFA strip in communication with the microfluidic network. In a preferred aspect, the test strip includes a capture probe, stripped to the strips surface and configured to bind to a target analyte. In another preferred aspect, the test strip is in communication with a passive pump configured to generate capillary-driven flow through the microfluidic network. In another preferred aspect, the passive pump of the invention may be a waste pad configured to generate capillary-driven flow through the microfluidic network.

In another aspect, the fluid washes the detection area prior to the first rehydrated reagent contacts the detection area. In alternative aspects, additional buffers may be introduced to the microfluidic network through the sample inlet.

In one aspect of the present invention includes systems and apparatus for sensing a chemiluminescent reaction including a printed circuit board, and a sensor configured as a plurality of pixels configured to detect photons, where the plurality of pixels are arranged on the sensor in a plurality of pixel islands. The system and apparatus for sensing a chemiluminescent reaction can further include an activation switch, as well as one or a plurality of circuitry elements in electrical communication with the sensor and activation switch. The system and apparatus for sensing a chemiluminescent reaction can further include a processor configured to send and receive electrical signals to and from the sensor, the activation switch and the circuitry elements. In a preferred aspect, each of the plurality of pixels of the sensor has at least one dimension equal to or greater than 2.0 micrometers (μm) and less than or equal to 10.0 micrometers (μm).

In one aspect of the present invention includes systems and apparatus for sensing a chemiluminescent reaction including a printed circuit board, and a sensor configured preferably as as a plurality of pixels configured to detect photons. In this aspect the plurality of pixels can be arranged on the sensor in a plurality of pixel islands. The system and apparatus for sensing a chemiluminescent reaction can further include an activation switch, as well as one or a plurality of circuitry elements in electrical communication with the sensor and activation switch. The system and apparatus for sensing a chemiluminescent reaction can further include a processor configured to send and receive electrical signals to and from the sensor, the activation switch and the plurality of circuitry elements. In a preferred aspect, each of the plurality of pixels of the sensor has at least one dimension equal to or greater than 3.0 micrometers (μm) and less than or equal to 7.0 micrometers (μm). In further aspects, the activation switch is an open electrical circuit that is closed upon introduction of a liquid sample to the apparatus, and the processor is a custom state machine.

### BRIEF DESCRIPTION OF THE FIGURES

FIG. 1 is a schematic illustration of an enzyme linked immunosorbent assay (ELISA).

FIG. 2 is a schematic illustration of multiple embodiments of a chemiluminescent microfluidic assay device 200 according to the present disclosure.

FIG. 3 shows an exemplary chemiluminescent microfluidic assay device 300 formed from multiple layers including from alternating layers of film (e.g., film layers 718A, 718B) and double-sided adhesive (e.g., double-sided adhesive layer 720).

FIG. 4A-E shows photographs of a prototype assay device consistent with the assay device of FIG. 2 during operation of the prototype assay device, with each including a respective illustration showing a state of a test strip.

FIG. 5 shows a flow chart illustrating a method of performing an assay requiring separate introduction of a sample and buffer according to the present disclosure.

FIG. 6 shows a flow chart illustrating a method of performing an assay of a combined sample/buffer fluid.

FIG. 7A-C shows (A) CI signal detection sensitivity and signal-to-noise ratios (SNR) at exemplary concentrations on a test strip in one embodiment thereof. (B) comparison of signal identify and detectability of an exemplary CI immunoassay run on a lateral flow strip compared to chemiluminescent microfluidic assay device configured to detect nucleocapsid (NP) protein from a SARS-coV-2 viral analyte, each samples run with 1,100 PFU/mL; (C) shows average ROI pixel intensity for testing and background averages over time and test line to background SNR over time, measures using a chemiluminescent microfluidic assay device of the invention.

FIG. 8A-B show experimental results for the detection of nucleocapsid protein (NP) from a fluid sample spiked with 100 ng/mL of recombinant nucleocapsid from SARS-CoV-2, a using monoclonal anti-NP from SARS-CoV-2 as a capture probe, monoclonal anti-NP antibody labeled with HRP as an enzyme label, and a luminal detection reagent in the presence of a peroxide catalyst.

FIG. 9 how experimental results for the detection of C-Reactive Protein (CRP) from a fluid sample spiked with 100 ng/mL of recombinant C-Reactive Protein (CRP), a using monoclonal anti-CRP as a capture probe, m polyclonal anti-CRP antibody labeled with ALP as an enzyme label, and a ECL reagent.

FIG. 10 demonstrates measured light intensity as a function of assay run time using a chemiluminescent microfluidic assay device.

FIG. 11A-E shows (A) exemplary images of test strip showing CI reaction following sample addition. (B) quantification of image data from (A), showing consistent background level detection, with signal saturation staying consistent over time-course (˜10 minutes). (C) quantification of image data of CI assay run at a lower concentration, showing positive measurement for over 7 minutes despite changes in intensity. (D, E) visual CI images and measurements from the experiment demonstrating signal intensity at the control and test lines over time.

FIG. 12A-B shows (A) ROI Averages and (B) ANR Averages for test line region 180 CFU/mL

FIG. 13 shows CI assays for clinical samples using dry swabs containing the sample that were added to a extraction buffer within our extraction tube, swirled 10×, filtered, and then the sample is transferred to the test cassette which is then read by the reader.

FIG. 14 illustrates a chemiluminescent response according to an exemplary embodiment of the present disclosure.

FIG. 15 shows a multiplex 1500 CI assay for clinical diagnosis of RSV 1501, Flu A 1502, Flu B 1503, COVID 1504.

FIG. 16A-C(A) shows the top half of a Capillary Driven Immunoassay (CaDI) housing 1600 with a preferred embodiment of the sample inlet port 1601 which splits the sample into two distinct pathways within the CaDI sample inlet. (B) shows alternate embodiments of the sample inlet port, each dividing the sample into a different number of aliquots, two 1601 or four 1602, or an unpreferred one 1603 for interaction with different pathways within the CaDI inlet port. (C) shows a cross section 1604 of a preferred embodiment of the sample inlet port depicted in 16A to show the relation of the sample inlet port 1605 to the CaDI sample inlet 1606.

FIG. 17 shows an assembly of one embodiment of the chemiluminescent CaDI reader/cartridge system 1700 comprised of a cartridge housing a chemiluminescent CaDI 1701, a camera with next generation back-thinned back-illuminated stacked CMOS light sensor 1702, a light guide 1703, a control system 1704 comprising a PCB comprising an image processor and control hardware, and a 2D code reader 1705.

FIG. 18 shows an alternate embodiment of the chemiluminescent CaDI reader-cartridge 1800 wherein a sensor 1801 (e.g. a CMOS sensor) is integrated into a printed circuit board 1802 comprising a processer 1803, memory, sensor control hardware 1804, and battery 1805 integrated into the CaDI Cartridge whereby the CaDI interfaces directly with sensor 1801 at nitrocellulose layer 1806 and backing layer 1807 via a fiber optic plate 1808 allowing for lossless translation of photons from the control 1809 and sample 1810 regions to the CMOS.

FIG. 19 illustrates aspects of sensors according to exemplary embodiments of the present disclosure.

FIG. 20 A-D depicts different orientations of a test strip, light guide, and sensor within a test cassette as well as signal generated from each orientation.

FIG. 21A-B depicts aspects of a preferred custom sensor at bottom (B) in comparison to commercially available camera sensor at top (A).

FIG. 22 depicts an exemplary embodiment of the test cassette.

FIG. 23A-D depicts (A, B) (C, D) 2 embodiments of a custom sensor developed for use with the testing cassette.

FIG. 24A-B depicts two embodiments of the control system of the test cassette. (A) An embodiment of the control system with components integrated into the sensor chip. (B) An embodiment of the control system with separate components placed on the PCB.

FIG. 25 depicts the series of steps for the function of the test cassette following emission of the chemiluminescent signal from the test strip.

FIG. 26 depicts an embodiment of the control system of the test cassette including a flexible PCB coupled with an adhesive.

FIG. 27A-C depicts (A, B, C) alternative applications for the custom sensor and control board described.

FIG. 28 shows the signal to background ratio for the CaDI system relative to a traditional Lateral Flow Assay (LFA) using chemiluminescence as the read out.

FIG. 29A-C. shows a comparison the CaDI platform (A) using chemiluminescence detection compared to two commercial products from Quidel®, the QuickVue™ (B) and the Sofia+™ (B).

FIG. 30. shows results of a competitive immunoassay for a small molecule target having a molecular weight is 296 g/mol using a CaDI device in one embodiment thereof.

### DETAILED DESCRIPTION OF THE INVENTION

Traditional enzyme linked immunosorbent assays (ELISAs) are used to detect a wide range of analytes with good sensitivity and specificity. These analytes include whole cells, proteins, antibodies, and small molecules, among other things. The high sensitivity of ELISAs is enabled by stringent washing steps to mitigate non-specific adsorption of non-targets and using an catalytic label such as an enzyme to amplify a signal, while the high specificity is enabled using a “sandwich” immunoassay that captures an analyte between two highly specific probes.

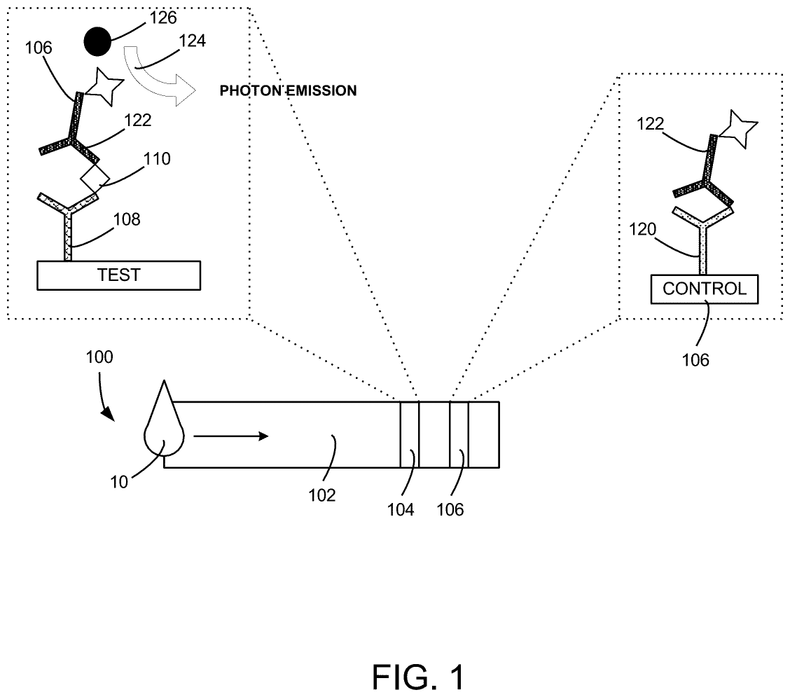

As shown generally in FIG. 1, a conventional ELISA system 100 includes a strip 102 that may have one or more detection zones or lines. In the specific example of this conventional ELISA system 100, the lines include a test line 104 and a control line 106. The strip 102 may be formed from nitrocellulose or a similar material such that, when a sample 10, is disposed on strip 102, sample 10 is wicked or otherwise transported to test line 104 and control line 106. A control line 106 may be optional but is generally included in ELISA systems to verify proper device functionality. In general, control line 106 includes an anti-label 120 adapted to bond with an enzyme label 122. Test line 104, on the other hand, includes a capture probe 108 configured to bond with a target analyte 110, to which enzyme label 122 bonds. Following delivery of target analyte 110 and enzyme label 122, an enzyme substrate 124 is provided that react, in the presence of a catalyst 126 with enzyme label 122 to produce a product and a corresponding chemiluminescent change of the corresponding line.

Both enzymatic detection and the sandwich assay format require multiple washing steps and sequential delivery of reagents (e.g., the enzyme label, substrate, and catalysts). These washing and reagent delivery steps yield robust analytical performance from ELISAs, but they are complicated to perform and relegate the assay to a centralized laboratory with expensive equipment and trained laboratory technicians. In many testing situations the time and financial resources needed for ELISAs are not available. There are more affordable point-of-care alternatives, but they often lack the analytical performance needed.

One of the most common alternatives to an ELISA is the lateral flow immunoassay. Lateral flow strips are inexpensive and easy to use in comparison to the ELISA. They generally require only that the end user provide a sample and the results are easy to interpret in most settings. The pregnancy test is the most widespread example of a lateral flow device. Although they are easy to use, lateral flow strips perform poorly compared to ELISAs, with well-documented reports of high false positive and negative rates and unreliable quantification. Substandard sensitivity and specificity in lateral flow assays stem from the inability to use enzymes as labels and sequentially wash and add reagents, which limits the scope of possible target analytes (e.g. disease biomarkers, antibodies, whole cells).

To address the foregoing issues, among others, this disclosure introduces a chemiluminescent microfluidic assay device to sequentially add and wash reagents and catalysts from a detection area to produce a chemiluminescent signal with minimal steps by an end user. The chemiluminescent microfluidic assay device generally includes a microfluidic network with channels that are specifically configured to provide sequential flow of reagents. catalysts and washing fluids to a detection area, also referred to as a detection zone. Operation of the chemiluminescent microfluidic assay device is substantially automatic, with an end user only being required to provide a sample to the device. In certain implementations, flow through the device is achieved by capillary driven flow, which may be facilitated by hydrophilic channels of the microfluidic network and a passive pump mechanism, such as a paper/microcellulose pump. Notably, chemiluminescent microfluidic assay devices according to the present disclosure are capable of performing tests similar to conventional ELISAs, enabling quick and accurate detection of target analytes in at-home and other settings and by untrained users that were previously undetectable outside a centralized laboratory.

Aspects of the present disclosure are directed to capillary-driven microfluidic devices and, more specifically, such devices for performing chemiluminescent testing. In general, the devices disclosed herein include a microfluidic network through which fluid is transported by capillary action and that automatically sequences the delivery of reagents, and preferably reagents that produce a chemiluminescent reaction, and washes to a test/detection area, also referred to as a zone. Sequencing is achieved, at least in part, by varying the length, geometry and/or other flow driving characteristics of pathways through the microfluidic network. For example, the microfluidic network of the invention may include paths of varying length and convolutions between a buffer inlet and the detection zone such that fluid transported along longer paths is delivered to the detection zone after fluid transported along shorter paths. Dried reagents and/or optionally catalysts may be disposed along certain paths of the microfluidic network, or provided separately, for example as a dried catalyst disposed of on the detection zone, or separately provided in a buffer, such that the reagents may be rehydrated and delivered to the detection zone. The flow-driving characteristics of the paths (e.g., the geometry of the paths) relative to each other may therefore be used to control the timing and sequencing of delivery of the rehydrated reagents and catalysts. Flow through the devices and subsequent sequencing of reagent delivery to the detection zone is substantially automatic and generally requires only that a user provide each of a sample and buffer solution or a combined sample and buffer to the device, depending on the particular test to be conducted using the device.

The microfluidic network includes multiple channels/paths, each configured to transport fluid by capillary flow from one or more fluid inlets to a detection area. Certain of the channels may include dried reagents that are rehydrated by fluid transported through the channels for subsequent delivery to the detection area. Other channels may not include reagents such that fluid transported through such channels is delivered to the detection area without substantially altering the composition of the fluid. As a result, fluid transported through channels furnished with a dried reagent may be used to deliver the reagent to the detection area. Washes may be provided, for example, by fluid portions that do not include rehydrated reagent or that are provided by other paths/channels of the microfluidic network that do not include dried reagents, or by separate plugs of buffer added to the sample inlet introduced subsequent to the fluid sample. By varying the length, size, and similar characteristics of the channels of the microfluidic network and including other flow control mechanisms, delivery of fluid via different channels to the detection area may be sequenced. So, for example, delivery of reagents and washes may be alternated or otherwise sequenced to follow a particular testing protocol. Dried reagents, and preferably reagents that produce a chemiluminescent reaction, may be provided by conjugate release pads disposed along channels of the microfluidic network. Such pads may be formed, e.g., from glass fiber, nitrocellulose, or similar porous materials. Alternatively, dried reagent may be disposed along a given channel, e.g., by being dried onto a surface of the channel.

In certain implementations, the detection area may be provided by a nitrocellulose membrane, sometimes referred to as a test strip, connected to or otherwise in communication with the microfluidic network. The nitrocellulose membrane may contain a capture probe specific to the target analyte. The capture probe may be striped onto the nitrocellulose membrane to form a test line. During operation, an enzyme-labeled detection antibody specific to the target analyte may be delivered to the test line (e.g., by rehydrating a dried antibody disposed in a channel of the microfluidic network) followed by subsequent delivery of a substrate (e.g., by rehydrating a dried antibody disposed in a channel of the microfluidic network) that reacts with the enzymatic label, often in the presence of a catalyst, to produce a chemiluminescent signal at the test and control line.

The chemiluminescent assay device of the invention may include a sample inlet and related components for processing a sample having more than one analyte for testing, such as an optional filtration membrane for filtering the sample. When performing an assay based on whole blood, for example, the filtration membrane may be a plasma separation membrane and may be sealed over the sample inlet. In such cases, the assay device may further include a separate fluid/buffer inlet for introducing a fluid buffer to initiate subsequent testing of the sample. For less viscous or less complex sample matrices (e.g., nasal swab samples diluted in an extraction buffer) a single sample inlet/buffer inlet with no filtration membrane could be used. In such applications, a separate membrane or similar element may not be necessary, and the sample may also supply the buffer for the rest of the assay. In general, however, the same reagent addition and washing steps may be accomplished with single-inlet assay device as with multi-inlet devices by configuring the microfluidic network accordingly.

Example embodiments discussed herein focus on non-competitive chemiluminescent immunoassays; however, implementations of the present disclosure are not limited to such assays. Rather, this disclosure is intended to describe a more general chemiluminescent assay device that may be used in a range of applications. Stated differently, this disclosure is intended to describe a general chemiluminescent assay device capable of sequential delivery of reagents and washes to detect a plurality of distinct analytes, which may preferably be through a chemiluminescent detection assay. Although specific tests (including specific sample types, reagents, buffers, etc.) may be described, such tests should be considered illustrative only and non-limiting regarding other applications for the present disclosure. For example, while most examples discussed herein focus on testing of biological samples, such as one or more bodily fluids, assay devices according to this disclosure may be adapted for environmental or chemical testing (e.g., water testing).

Again, while the examples disclosed herein generally focus on non-competitive chemiluminescent immunoassays, the chemiluminescent assay devices according to the present disclosure may be adapted to perform competitive immunoassays. Competitive chemiluminescent immunoassays are more commonly used for small molecules, like hormones, THC, and other small molecular weight molecules where an antibody pair is not available. In a competitive immunoassay, a capture antibody is deposited in a detection area (e.g., on a nitrocellulose strip). A detection reagent in the system would be a chemiluminescent labeled version of the analyte. When the sample runs through the system with no analyte, the labeled detector analyte binds to the capture antibody and gives a signal, e.g., a chemiluminescent signal at the test line. If analyte is present in the sample, it competes with the detector analyte binding at the detection area and reduces the resulting chemiluminescent signal. Therefore, a reduction in signal is observed in the presence of specific analyte. So, for example, in implementations of the present disclosure, an assay device may be configured to deliver a capture antibody to a detection area followed by a sample, followed by a labeled version of a plurality of distinct analytes, with optional washes between each delivery.

In a preferred embodiment, the present invention includes systems, methods and devices directed to a novel chemiluminescent assay. In this preferred embodiment, the chemiluminescent assay device of the invention includes a chemiluminescent testing assembly further including a sample inlet for receiving a fluid sample in communication with a microfluidic network formed by a first and a second path extending to a detection area, wherein that detection area is configured to capture a target analyte, for example by a capture probe configured to bind to a target analyte in a fluid sample disposed of on the detection area. In this preferred embodiment, a first dried reagent is disposed along the first path, while a second dried reagent disposed along the second path. In this configuration, when a fluid is provided to the sample inlet the fluid fills the microfluidic network and directs a portion of the fluid through the microfluidic channel to the detection area where the target analyte, if present in the sample, is captured, and wherein a first portion of the fluid rehydrates the first dried reagent to produce a first rehydrated reagent, a second portion of the fluid rehydrates the second dried reagent to produce a second rehydrated reagent, and the first rehydrated reagent and the second rehydrated reagent are sequentially delivered to the detection area by capillary-driven flow, and wherein the first reagent, and the second reagent react in the presence of a catalyst, the reaction emitting an chemiluminescent signal. The sample inlet may include a filtration membrane in certain embodiments.

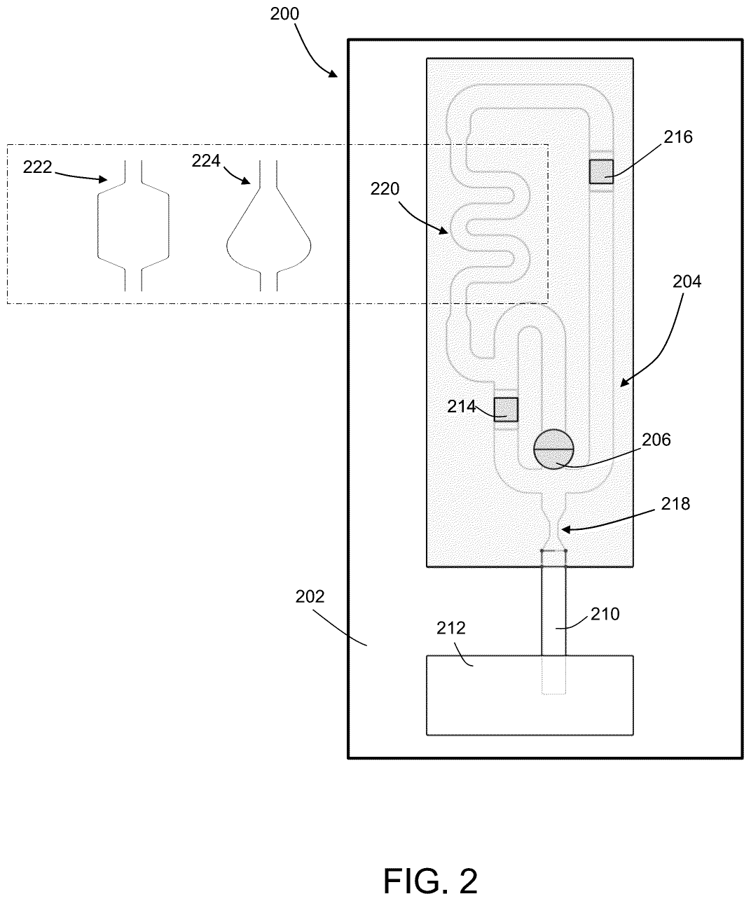

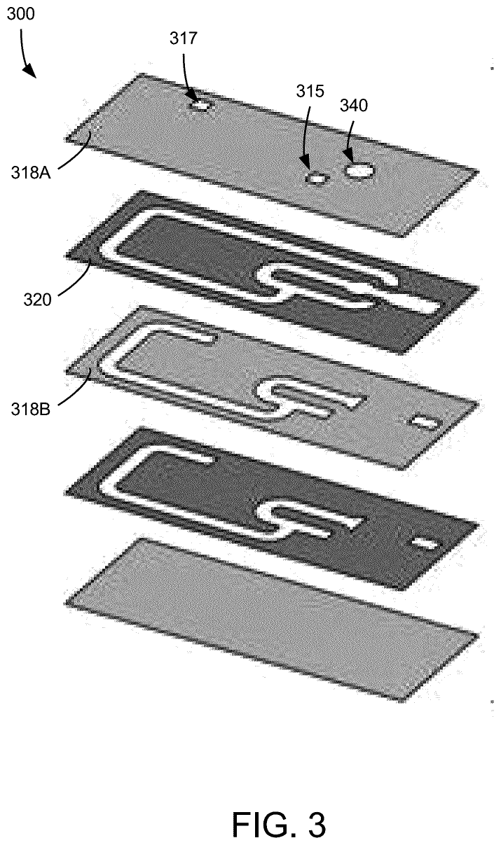

In certain embodiment, the chemiluminescent assay device is made of film sheets (e.g., transparency sheets, polyester film) and double-sided adhesive layers. Each layer may be laser cut or otherwise manufactured such that, when the layers are assembled by stacking and laminated, a microfluidic network is defined within the resulting laminated body. For example, as shown in FIG. 3, film layer 318A, which corresponds to a topmost layer of the chemiluminescent assay device 300, may define various openings. More specifically, film layer 318A includes each of an inlet opening 340 corresponding to a sample inlet 206 (shown in FIG. 2) as shown herein, and each of a first vent 315 corresponding to dried enzyme label pad 214 (shown in FIG. 2) and a second vent 317 corresponding to dried substrate pad 216 (shown in FIG. 2). As noted above in the context of the chemiluminescent assay device 200, vents 315, 317 generally facilitate filling of device body 202 with buffer fluid without formation of bubbles that may disrupt flow through microfluidic network 204.

Generally referring to FIG. 2, in one preferred embodiment the invention includes a chemiluminescent assay device 200 having a device body 202 defining a microfluidic network 204 and sample inlet 206 (also referred to generally herein as an inlet) in communication with microfluidic network 204. In general, microfluidic network 204 transports fluids provided via inlet 206 to a detection area 208 containing a test strip 210, which is shown as being coupled to a waste pad/passive pump 212. The chemiluminescent assay device 200 is generally configured to perform a test including enzyme labelling and subsequent application of a chemiluminescent substrate, the reaction of which causes the emission of a quantity of photons as an observable signal. Accordingly, the chemiluminescent assay device 200 includes a dried enzyme pad 214 and a dried chemiluminescent substrate pad 216 disposed within microfluidic network 204.

In the illustrated embodiment microfluidic network 204 comprises a delay element 220 configured to allow more time for excess elements transported from a dried enzyme pad 214 to move over and off of test strip 210 before the elements from chemiluminescent substrate pad 216 arrive at test strip 210. Delay element 220 is illustrated as a convoluted portion of microfluidic network 204, with multiple bends to increase the length of the microfluidic channel between dried enzyme pad 214 and chemiluminescent substrate pad 216. Other embodiments may comprise a delay element configured similar to delay element 222 or delay element 224, which include microfluidic channels with increased cross-sectional area to delay allow more time for excess elements transported from dried enzyme pad 214 toward chemiluminescent substrate pad 216. Accordingly, delay element 222 provides for a flow time through microfluidic network 204 from chemiluminescent substrate pad 216 to test strip 210 that is greater than the flow time from dried enzyme pad 214 to test strip 210. In the embodiment shown, centering element 218 is provided to ensure chemiluminescent substrate flows through the center portion of test strip 210 and not down the edges of test strip 210. In the illustrated embodiment, centering element 218 comprises a reduced (e.g. narrower) cross-section of the microfluidic channel in microfluidic network 204. The reduced cross-section of centering element 218 provides for an increased response signal in the center of test strip 210 allowing for increased accuracy and sensitivity when detecting results.

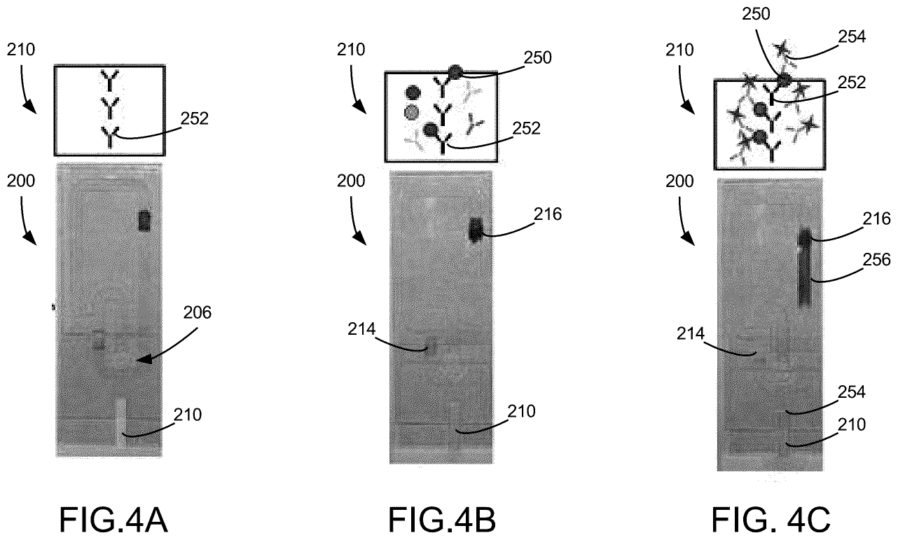

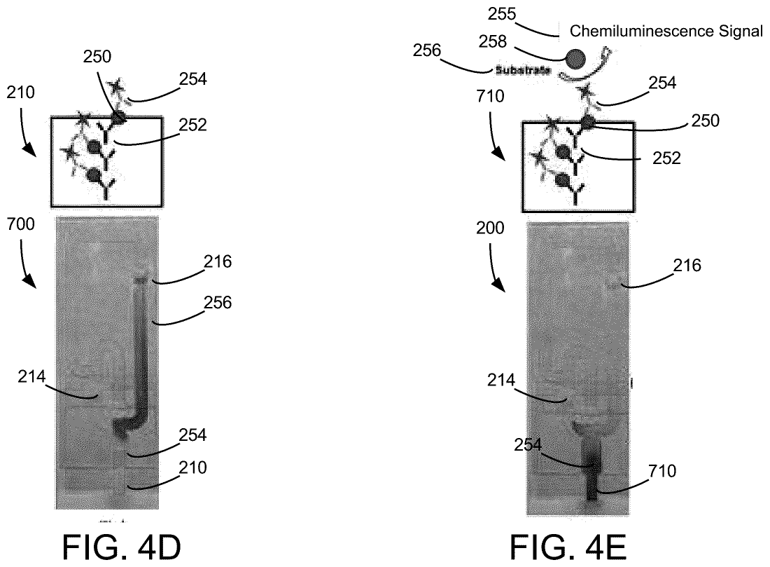

FIGS. 4A-E illustrate operation of the chemiluminescent assay device 200. More specifically, each of FIGS. 4A-E include a photograph of a chemiluminescent assay device 200 accompanied by a schematic illustration of test strip 210 that indicating the state of test strip 210 and any reagents/substances provided thereto. Notably, as would be understood by those of ordinary skill in the art, food coloring was used in place of actual enzyme labels and reagents to better illustrate operation of FIGS. 4A-E; nevertheless, the following example still refer to enzyme labels and substrates.

FIG. 4A illustrates chemiluminescent assay device 200 in a pre-assay state. In the pre-assay state, fluid has not yet been provided to inlet 206 and each of enzyme label pad 214 and dried substrate pad 216 include corresponding dried reagents. As illustrated in the detailed view of test strip 210, test strip 210 may include capture probes (such as capture probe 252) adapted to bond with a target analyte. As previously discussed, test strip 210 may include other control capture probes for purposes of testing and validating operation of chemiluminescent assay device 200.

FIG. 4B illustrates chemiluminescent assay device 200 following addition of a test fluid to inlet 206. In general, the fluid is distributed from inlet 206 to different channels of microfluidic network 204 by capillary action. Such distribution of the test fluid may result in the test fluid contacting dried enzyme label pad 214 and initiating rehydration of the dried enzyme label stored on dried enzyme label pad 214. Similarly, a portion of the test fluid may also contact dried substrate pad 216, initiating rehydration of a dried substrate. Yet another proportion of the fluid is transported to test strip 210 such that a proportion of a target analyte 250 contained within the test fluid binds with the capture probes 252. As illustrated in the detailed view of test strip 210 included in FIG. 4B, introduction of the test fluid may cause other analytes and trace amounts of the rehydrated enzyme label and rehydrated substrate to reach test strip 210.

FIG. 4C illustrates chemiluminescent assay device 200 following introduction of the test fluid and substantial elapsed time such that a substantial portion of the rehydrated enzyme label 254 is transported from dried enzyme label pad 214 to test strip 210. As further illustrated in FIG. 4C, a portion of rehydrated substrate 256 has simultaneously started moving toward test strip 210. Also, as illustrated in the detail view of test strip 210, at least a portion of rehydrated enzyme label 254 arriving at test strip 210 bonds with target analyte 250 previously captured at test strip 210, thereby preparing test strip 210 for delivery of rehydrated substrate 256. During delivery of enzyme label from dried enzyme label pad 114, an excess of enzyme label may be delivered to test strip 110. Accordingly, FIG. 4D illustrates chemiluminescent assay device 200 during a wash after delivery of rehydrated enzyme label 254. More specifically, microfluidic network 204 is generally shaped and configured such that at least a portion of the test fluid is delivered to test strip 210 after rehydrated enzyme label 254 but before rehydrated substrate 256. By doing so, excess enzyme label can be removed from test strip 210 before arrival of rehydrated substrate 256, generally improving the response of test strip 210.

FIG. 4E illustrates chemiluminescent assay device 200 during delivery of rehydrated substrate 256 to test strip 210. As shown in the inset of FIG. 4E, delivery of rehydrated substrate 256 results in the production of a chemiluminescent signal 255, which may, in turn, cause a chemiluminescent change in test strip 210, such as the detectable emission of light. Notably, in certain embodiments, the rehydrated substrate 256 may result in the production of a chemiluminescent signal in the presence of a catalyst 258. Catalyst 748 can be disposed of within the fluid, or within a buffer, or on the dried substrate pad, or on the test strip.

As noted above, the preset invention further includes method of performing a chemiluminescent assay. In this preferred embodiment, the method can include receiving a fluid at a sample inlet of an assay device, wherein the assay device includes a microfluidic network in communication with the sample inlet and a chemiluminescent assembly including a detection area configured to capture a target analyte. Flowing the fluid through the microfluidic pathway, initiating the steps of rehydrating a first dried reagent disposed along a first path of the microfluidic network to produce a first rehydrated reagent, and rehydrating a second dried reagent disposed along a second path of the microfluidic network to produce a second rehydrated reagent. An additional preferred step of the invention includes sequentially delivering the first rehydrated reagent and the second rehydrated reagent to the detection area by capillary flow and wherein the first reagent, and the second reagent react, optionally in the presence of a catalyst, the reaction emitting an chemiluminescent signal.

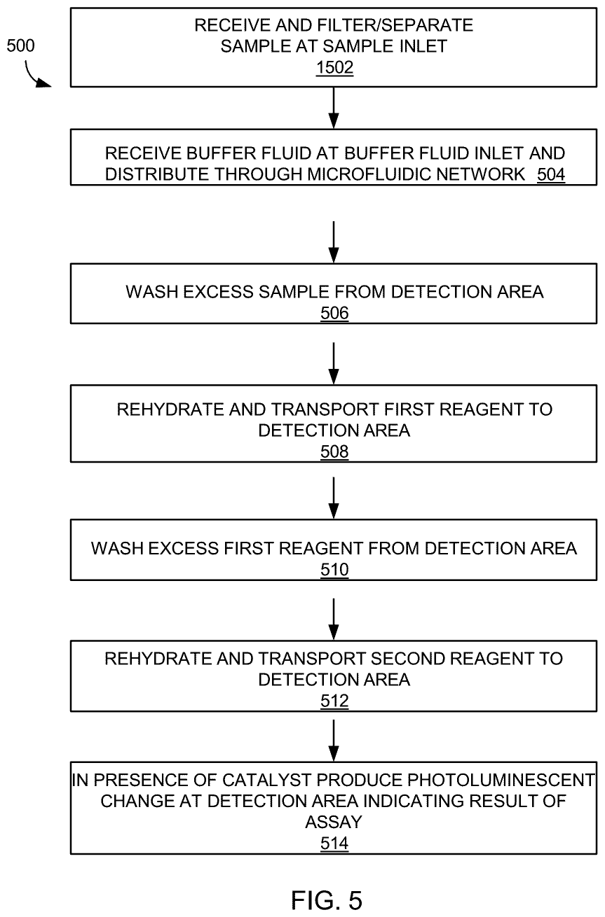

As further shown in FIG. 5, a flow chart of a first method 500 of performing an assay using a and assay device having multiple inlets is described. At operation 502, a sample is received at a sample inlet of an assay device. The assay device includes a microfluidic network in communication with each of the fluid inlet and a chemiluminescent assembly including a detection area. In certain implementations, the sample is filtered, e.g., using a filtration membrane. The sample may also be driven, at least partially, to the detection area by capillary-driven flow when the sample is introduced. At operation 504, a buffer fluid is received at a buffer fluid inlet of the assay device. The buffer fluid may substantially fill the microfluidic network and addition of the buffer fluid may generally initiate capillary-driven flow through the microfluidic network. Capillary-driven flow may also be facilitated by a passive pump in communication with the microfluidic network.

At operation 506, the buffer fluid flows across the detection area, washing excess sample from the detection area. At operation 508, a first rehydrated reagent is delivered to the detection area. More specifically, a first dried reagent, such as a dried enzyme label, may be disposed along a first path of the microfluidic network (e.g., in the form of a pad onto which the first reagent is dried). When the buffer fluid is added, the buffer fluid may rehydrate the first dried reagent and may initiate transportation of the first rehydrated reagent to the detection area. At operation 510, another portion of the buffer fluid may be transported across the detection area, washing excess first reagent from the detection area. At operation 512, a second rehydrated reagent is delivered to the detection area. More specifically, a second dried reagent, such as a dried substrate, may be disposed along a second path of the microfluidic network (e.g., in the form of a pad onto which the second reagent is dried). When the buffer fluid is added, the buffer fluid may rehydrate the second dried reagent and may initiate transportation of the rehydrated second reagent to the detection area.

The first and second dried reagents may react, optionally in the presence of a catalyst provided with the rehydrated substrate, in the buffer and/or sample fluid, or disposed on the detection area. The product of this reaction may include a chemiluminescent in the form of an emission of detectable photons. At operation 514, a result of the chemiluminescent assay is visually indicated. For example, by a detectable chemiluminescent indicator may appear at the detection area in response to a chemiluminescent signal produced by delivery of the substrate.

Notably, devices in accordance with the present disclosure are configured to perform operations 506-514 of FIG. 5 automatically in response to receiving each of the sample and buffer fluid. Stated differently, the washes and reagent deliveries occur automatically due to capillary-driven flow and, as a result, do not require intervention by an end user beyond introduction of the sample and the buffer fluid. While operations 508 (rehydration and delivery of first reagent) and 512 (rehydration and delivery of second reagent) are generally discussed above and illustrated as sequential, rehydration of the first and second reagents and initial transportation of the first and second reagents may be simultaneous or overlap following introduction of the buffer fluid. However, the microfluidic network is configured such that the rehydrated second reagent arrives at the detection area after the first reagent and, in most applications, after a wash performed after delivery of the first reagent to the detection area.

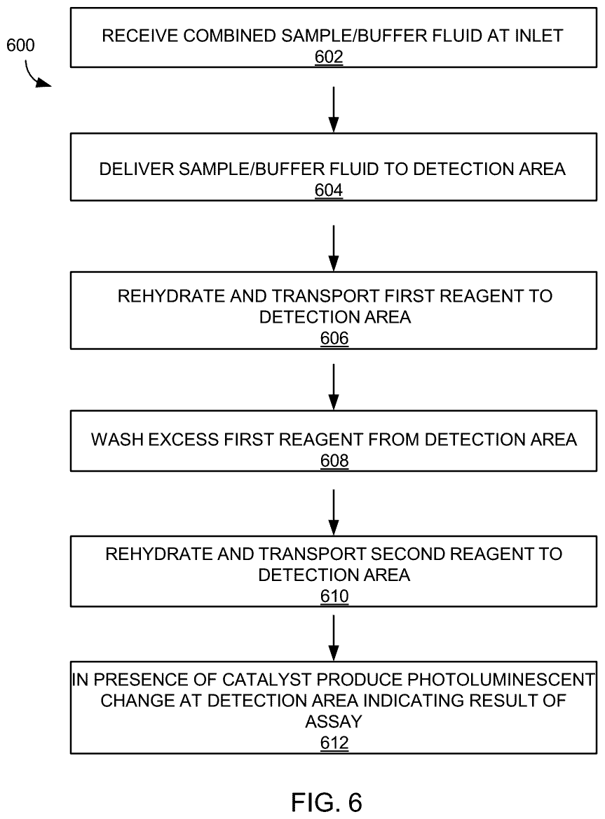

FIG. 6 is a flow chart of a second method 600 of performing a chemiluminescent assay using a single inlet assay device, such as assay device 200 of FIG. 2. At operation 602, a fluid sample, and preferably a combined sample and buffer fluid is received at a fluid inlet of an assay device. The assay device includes a microfluidic network in communication with the fluid inlet and a chemiluminescent assembly including a detection area. The sample/buffer fluid may substantially fill the microfluidic network and addition of the sample/buffer fluid may generally initiate capillary-driven flow through the microfluidic network. Capillary-driven flow may also be facilitated by a passive pump in communication with the microfluidic network. At operation 604, the sample/buffer fluid flows across the detection area, delivering sample to the detection area, where a target analyte in the fluid can bind to a capture probe.

At operation 606, a first rehydrated reagent is delivered to the detection area. More specifically, a first dried reagent, such as a dried enzyme label, may be disposed along a first path of the microfluidic network (e.g., in the form of a pad onto which the first reagent is dried). When the sample/buffer fluid is added, the sample/buffer fluid may rehydrate the first dried reagent and may initiate transportation of the resulting rehydrated reagent to the detection area. At operation 608, another portion of the sample/buffer fluid may be transported across the detection area, washing excess first reagent from the detection area.

At operation 610, a second rehydrated reagent is delivered to the detection area. More specifically, a second dried reagent, such as a dried substrate, may be disposed along a second path of the microfluidic network (e.g., in the form of a pad onto which the second reagent is dried). When the sample/buffer fluid is added, the sample/buffer fluid may rehydrate the second dried reagent and may initiate transportation of the rehydrated second reagent to the detection area. The first and second dried reagents may react, optionally in the presence of a catalyst provided with the rehydrated substrate, in the buffer and/or sample fluid, or disposed on the detection area. The product of this reaction may include a chemiluminescent in the form of an emission of detectable photons. At operation 612, a result of the assay is visually indicated. At operation 514, a result of the chemiluminescent assay is visually indicated. For example, by a detectable chemiluminescent indicator may appear at the detection area in response to a chemiluminescent signal produced by delivery of the substrate.

Again, chemiluminescent devices in accordance with the present disclosure are configured to perform operations 604-612 of FIG. 6 automatically in response to receiving the sample/buffer fluid. Stated differently, the washes and reagent deliveries occur automatically due to capillary-driven flow and, as a result, do not require intervention by an end user beyond initial introduction of the sample/buffer fluid. While operation 606 (rehydration and delivery of first reagent) and operation 610 (rehydration and delivery of second reagent) are generally discussed above and illustrated as sequential, rehydration of the first and second reagents and initial transportation of the first and second reagents may be simultaneous or overlap following introduction of the sample/buffer fluid. However, the microfluidic network is configured such that the rehydrated second reagent arrives at the detection area after the first reagent and, in most applications, after a wash performed after delivery of the first reagent to the detection area.

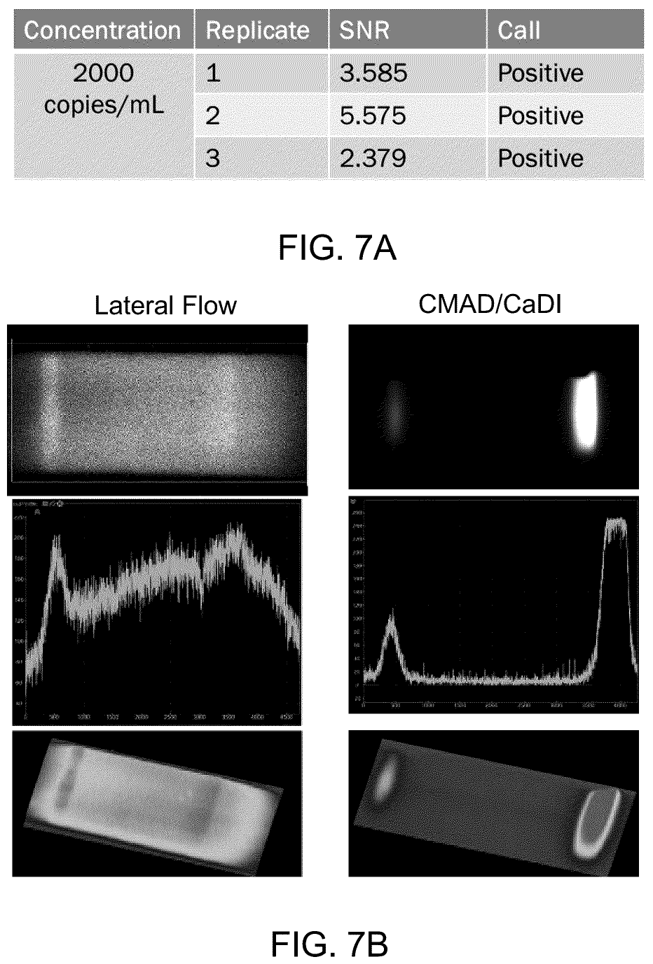

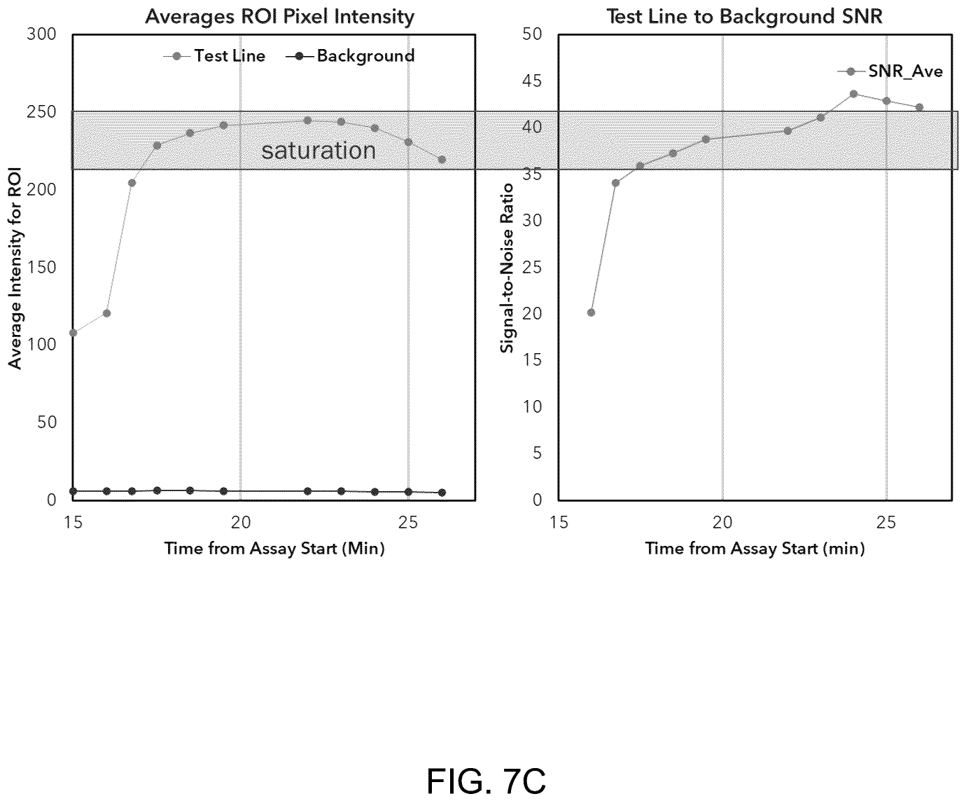

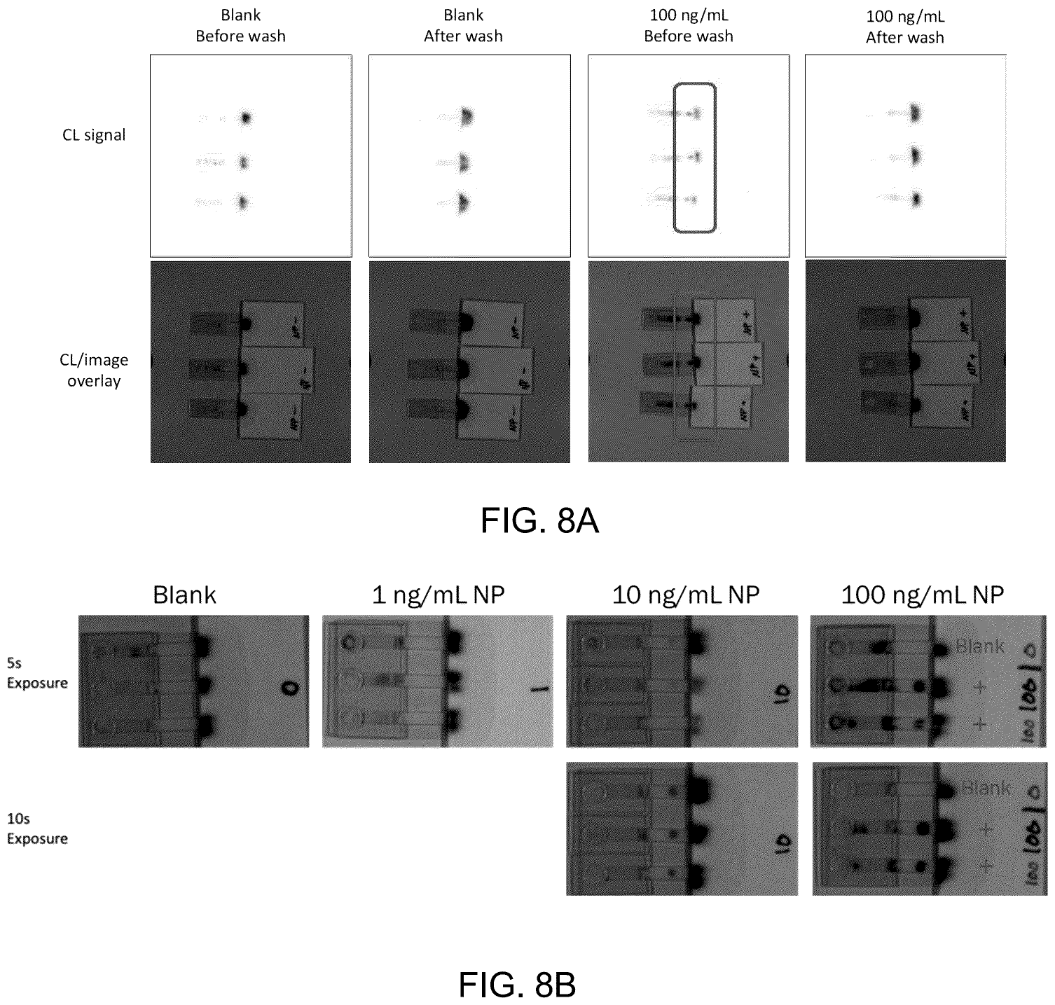

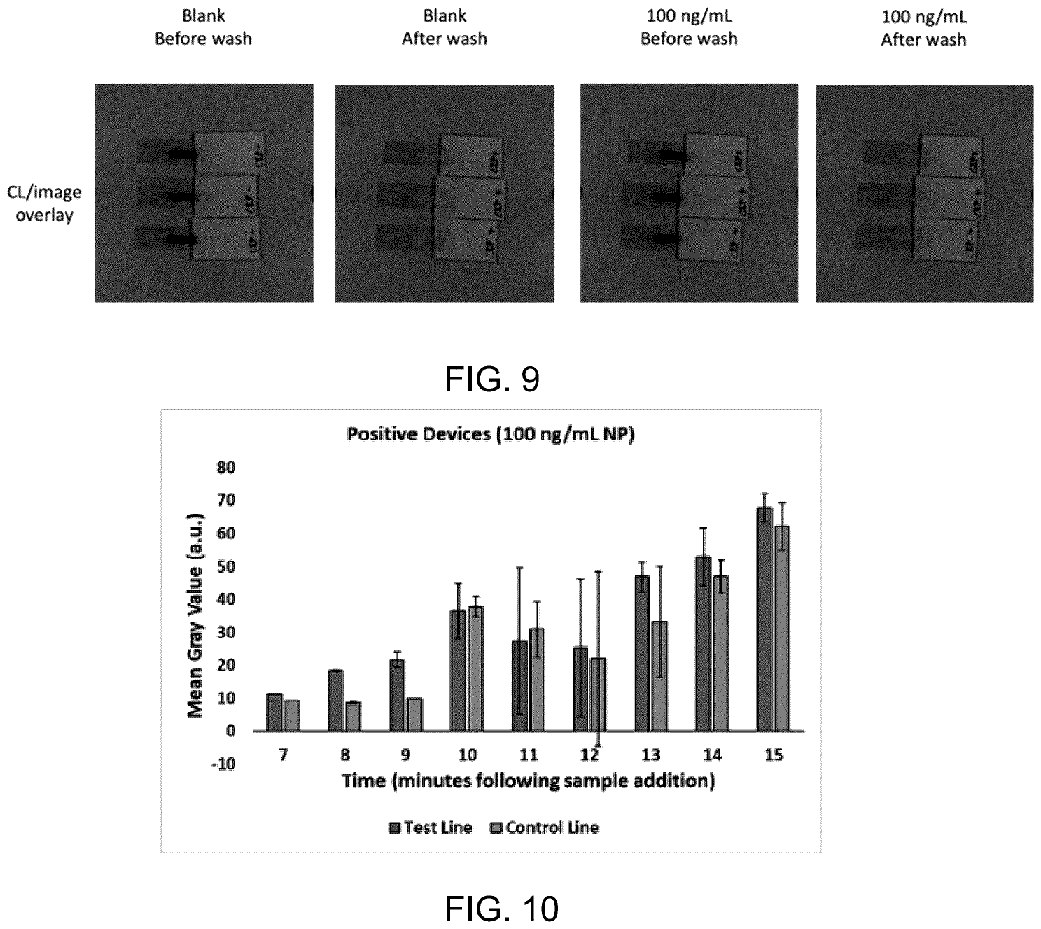

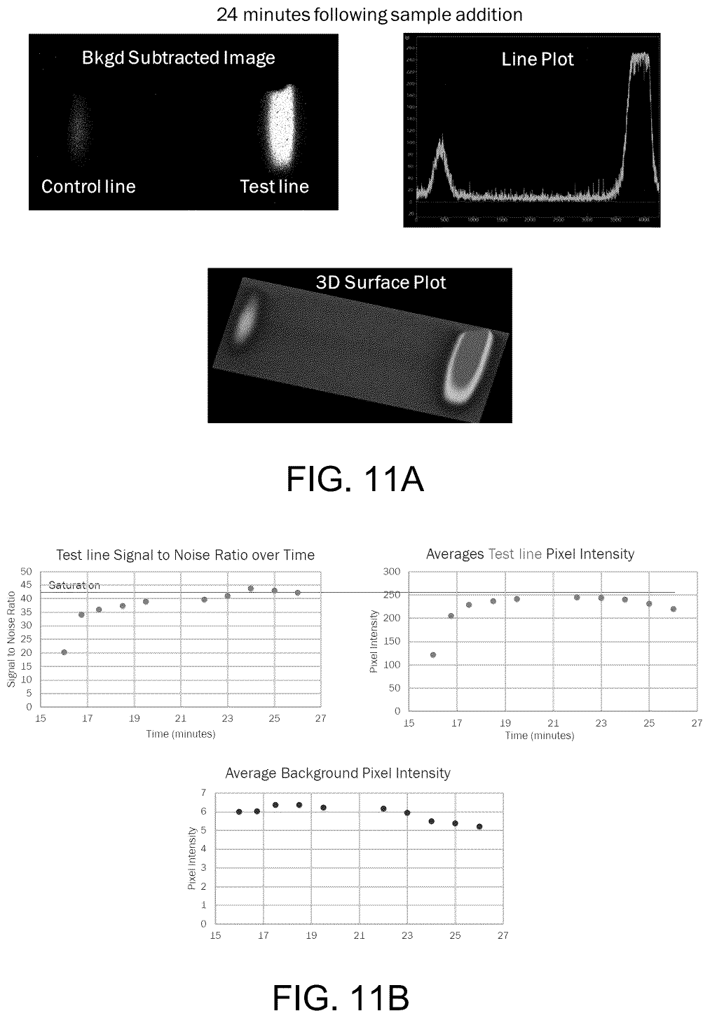

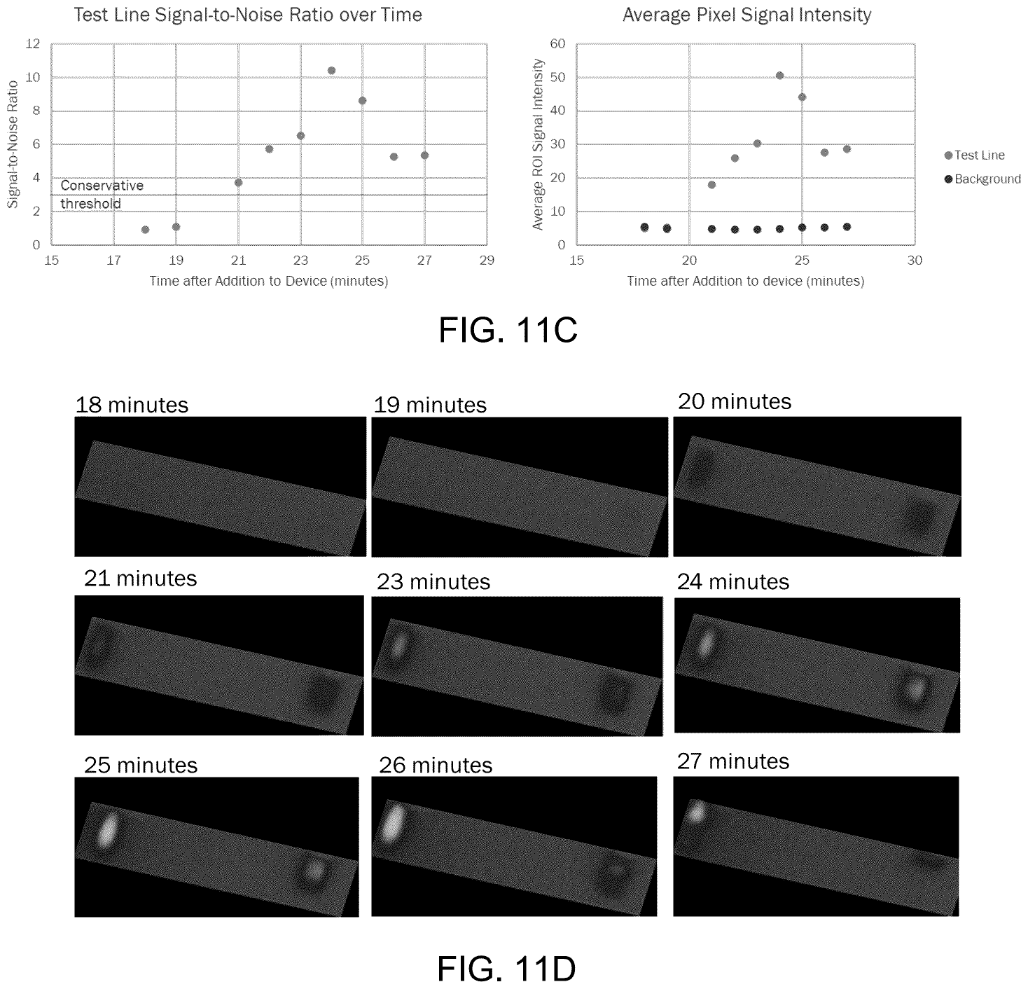

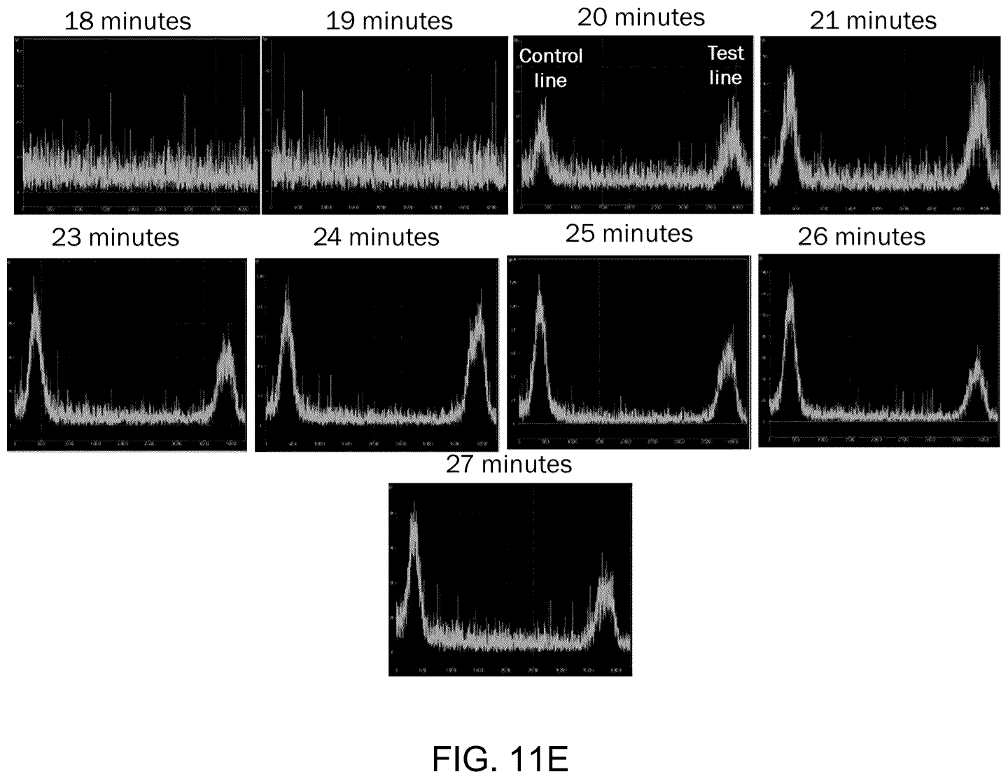

As shown in FIG. 7A, the chemiluminescent devices in accordance with the present disclosure provide a signal to noise ratio (SNR) for three replicate measurements at 2000 gene copies/mL sample concentrations for SARS-CoV-2. As further shown in FIG. 7B, the left column shows results for a lateral flow assay performed in an exemplary chemiluminescence mode. The top image of the figure is a full emission image for the detection zone of the lateral flow assay. The middle image demonstrates a line scan horizontally through the top image showing the features. The bottom image is a heat map view of the top image, while the right column results show the same experiment for the CaDI system. Further, the top image of the Figure shows the chemiluminescence output in the detection assay in the readout window. The middle image shows a line scan horizontally through the top image. The bottom image is a heat map of the top image. As further demonstrated in FIG. 7C, responses on CaDI (left) versus lateral flow (right) in terms of signal to noise ratio are compared. As shown in FIG. 8, the present inventors demonstrate (A) photographs of results at different stages of the chemiluminescent assay, and at (B) different concentrations. FIG. 9 provides additional photographs of an exemplary chemiluminescent assay at different steps of the assay. FIG. 10 shows a signal as a function of time for positive tests at 100 ng/mL of the N-protein showing both test and control lines of an exemplary chemiluminescent assay. As further shown in FIG. 11, an exemplary chemiluminescent assay is demonstrated showing (A) a background subtract image of test results (top left), a line scan plot for the first image (top right), and a heat map of the subtracted image (bottom). FIG. 11B shows a comparison of signal noise, test line intensity, and background intensity as a function of time for an exemplary chemiluminescent assay. FIG. 11C shows (Left) a signal to noise ratio over time relative to a potential conservative detection threshold, as well as (Right) an average pixel intensity over time for test line and background. FIGS. 11D-E show heat maps, and line scans for different time points of exemplary chemiluminescent assay experiments.

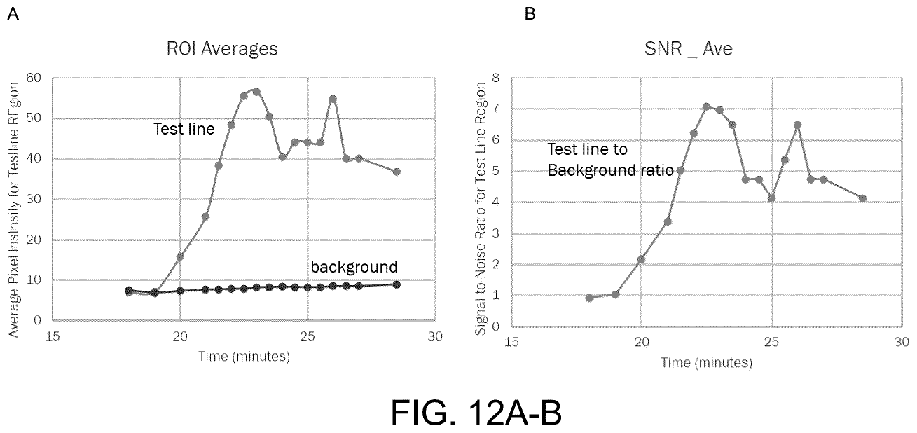

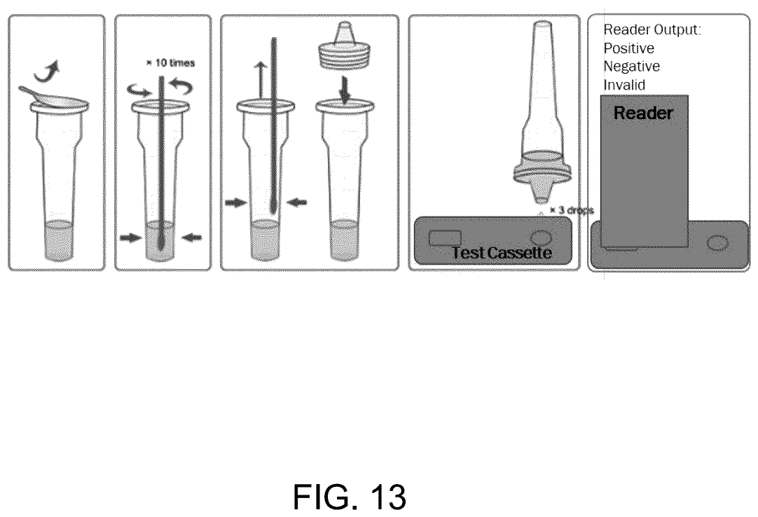

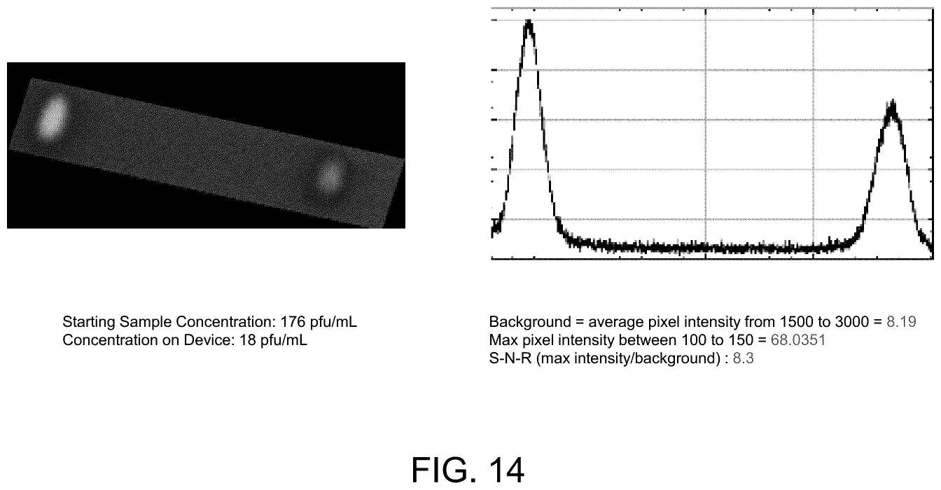

FIG. 12 (Left) shows the comparison of ROI averages for a test line and background, and (Right) Signal to noise ratio test line vs background in one exemplary test of the chemiluminescent assay of the device. As noted above, FIG. 13 demonstrates an exemplary schematic representation of the chemiluminescent assay steps of the invention in one embodiment. FIG. 13B (Left) shows a comparison of CaDI to a lateral flow assay using the same samples, including (Right) images from the actual data analysis at 1000 PFU/mL concentrations. FIG. 14 demonstrates example results for low concentration samples, 176 PFU/mL in the sample and 18 PFU/mL added to the device of the invention.

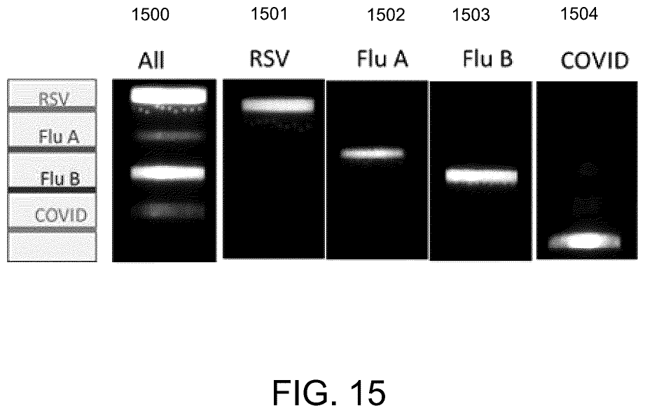

Chemiluminescent devices can be multiplexed as shown in FIG. 15, RSV 1501, Flu A 1502, Flu B 1503 and COVID 1504 are present on one nitrocellulose strip 1500 and are visible with minimal cross reactivity or background signal. The multiplex assay depicted in FIG. 15 represents but one embodiment of a multiplex assay that could be developed with the CaDI device. Alternate embodiments of the invention may analyze different viruses, bacteria, parasites, fungi, or other analytes in a multiplexed assay. In some cases, different Capillary Driven Immunoassay (CaDI) devices may be tailored to aid in diagnosis of specific symptoms or disease states, such as symptoms associated with respiratory viruses.

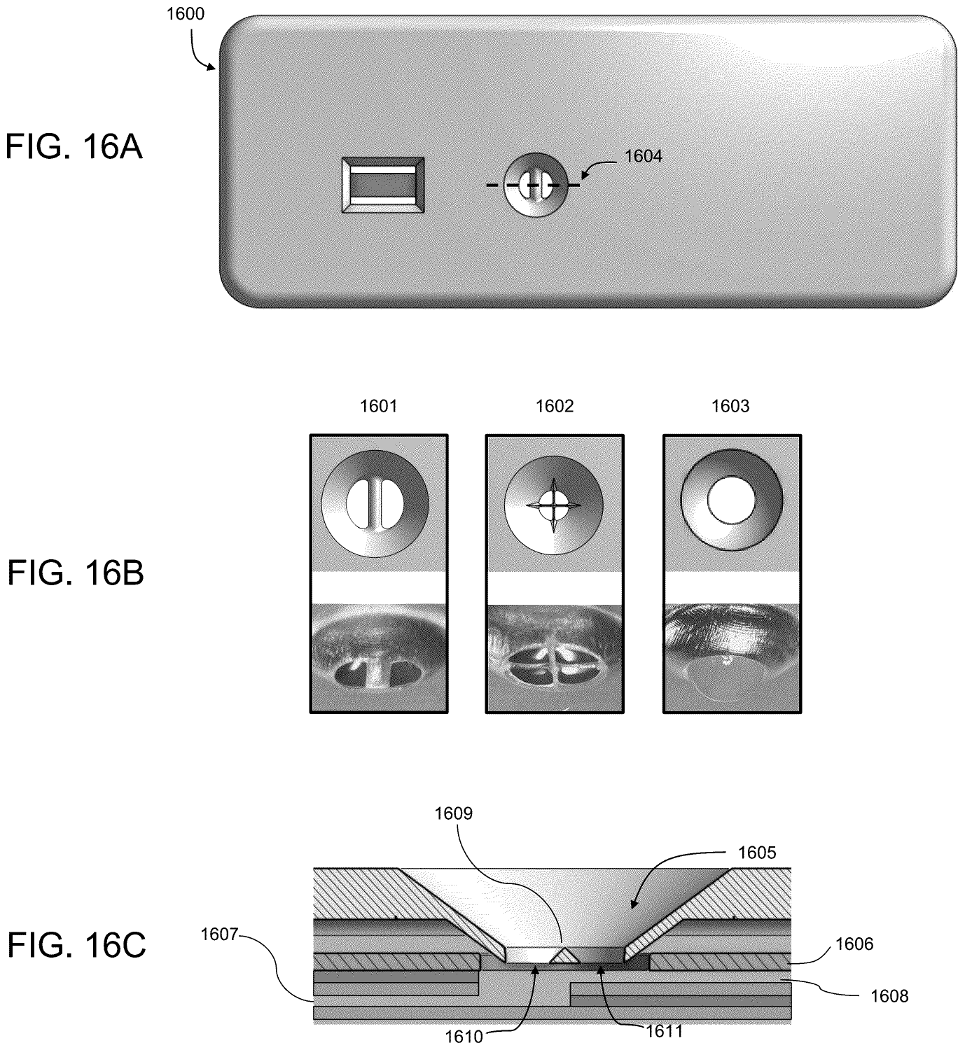

Addition of a sample to the CaDI through a sample inlet port 1605 within top portion 1600 of the CaDI cartridge is important to successfully running an assay. In order for the CaDI to run correctly, the sample should simultaneously (or nearly simultaneously) contact multiple hydrophilic surfaces within the CaDI sample inlet. In one embodiment of the sample inlet (206) there are two surfaces, 1607 and 1608, for the liquid sample to make contact with. In alternate embodiments, the liquid sample may make contact with 1, 2, 3, 4, or more surfaces simultaneously. To ensure the sample contacts both surfaces independently and reliably, an embodiment 1601, shown in FIG. 16B, of the sample inlet port can be used in order to direct the sample in two directions simultaneously (or nearly simultaneously). FIG. 16C further describes cross section 1604 of the inlet port in an embodiment. In this embodiment, a triangular bridge feature 1609 bisects inlet 1605 thereby separating liquid droplets into two halves, 1610 and 1611. The sample divided into 1610 makes contact with the first surface, 1607, while the sample divided into 1611 makes contact with the second surface 1608. While the illustrated embodiment of the bisecting bridge feature 1609 uses a triangular shape, alternate embodiments may use different shape profiles to achieve similar division of the liquid as seen in the preferred embodiment of the inlet port 1605. Alternate embodiments may also remove the bridge feature 1609 as seen in alternate inlet embodiment port 1603. In yet other embodiments, the bridge feature 1609 may be replaced with alternate designs to facilitate division of the sample into more than two parts, as seen in the embodiment of the inlet port 1602 that would divide the sample into four equal parts.

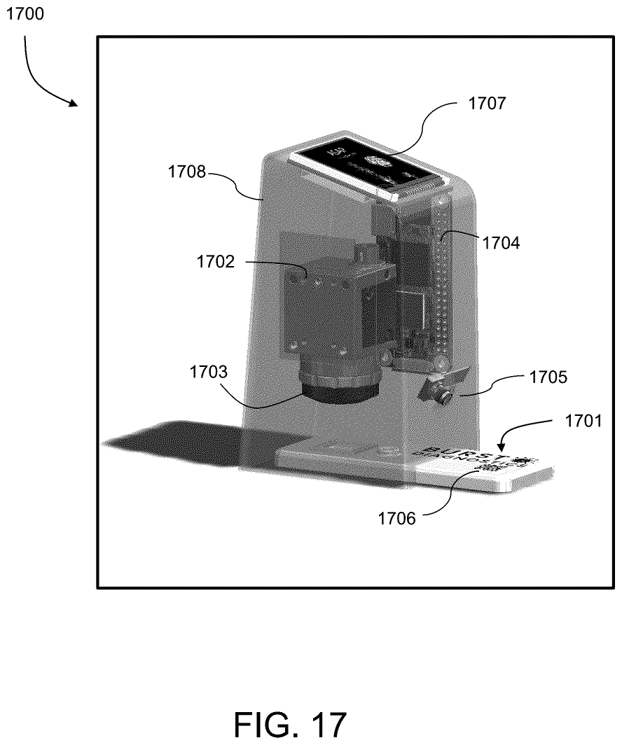

FIG. 17 illustrates a cartridge-reader system 1700 and the individual components in the system. In this embodiment, the CaDI cartridge 1701 is a plastic housing containing and protecting the CaDI described previously in FIG. 2. In some embodiments, the CaDI cartridge may include a code 1706 to identify individual CaDI cartridges. In one embodiment, a 2D code is applied to the CaDI cartridges. In other embodiments, the identification code 1706 may be a linear barcode instead of a 2D code. In yet other embodiments, the CaDI cartridge may omit a code in favor of some other identification method. In the illustrated embodiment, the reader comprises a housing 1708, which can be injected molded, and further contain a combination of components including a sensor 1702, a light guide 1703, a control system 1704. In certain embodiments control system 1704 comprises a printed circuit board containing processors capable of storing and running machine vision software, a touchscreen display 1707, and a QR/barcode/text recognition camera 1705. Together, these components comprise an example of a chemiluminescent assay reader system. In the embodiment shown, light guide 1703 is configured to direct a chemiluminescent response from the chemiluminescent assay device in CaDI cartridge 1701 to sensor 1702. In certain embodiments sensor 1702 can be configured to receive a chemiluminescent response from a chemiluminescent assay device (e.g. CaDI cartridge 1701) directly without a lens. Control system 1704 can then analyze the response detected by sensor 1702 and provide appropriate feedback to a user. For example, control system 1704 can indicate the presence or absence of an analyte in a sample contained in CaDI cartridge 1701.

An exemplary embodiment of the CaDI cartridge was used with an embodiment of the reader in FIG. 17, and the results were compared against commercially available tests for SAR-CoV-2 using antigen concentrations ranging from 0 ng/ml to 1000 ng/ml. Results indicated that a standard colorimetric was the least sensitive indicating positive detection of the antigen at 10 ng/ml. A commercially available fluorescent immunoassay (FIA), which uses fluorescent markers and an external reader to detect fluorescence, was also evaluated and found to have a detection limit of 5 ng/ml. By contrast the CaDI cartridge in combination with the reader had a detection limit of 0.1 ng/ml. Based on the evaluations, the CaDI cartridge is 100 times more sensitive than the commercially available colorimetric assay, and 50 times more sensitive than the FIA.

Referring again to the reader in FIG. 17, incorporating the camera sensor, or another Complementary Metal Oxide Semiconductor (CMOS) sensor, and control systems into the CaDI cartridge would allow for performing the chemiluminescent assay without the external reader. This integrated sensor test cassette, also referred to in this disclosure as the “test cassette,” improves upon the CaDI cartridge and external reader design mentioned earlier by incorporating the reader's sensor and the CaDI cartridge, thereby creating a single integrated cassette, thereby eliminating the need for an external reader. The test cassette could be used to perform chemiluminescent assays in remote locations, or in at-home settings where a reader might not be readily available.

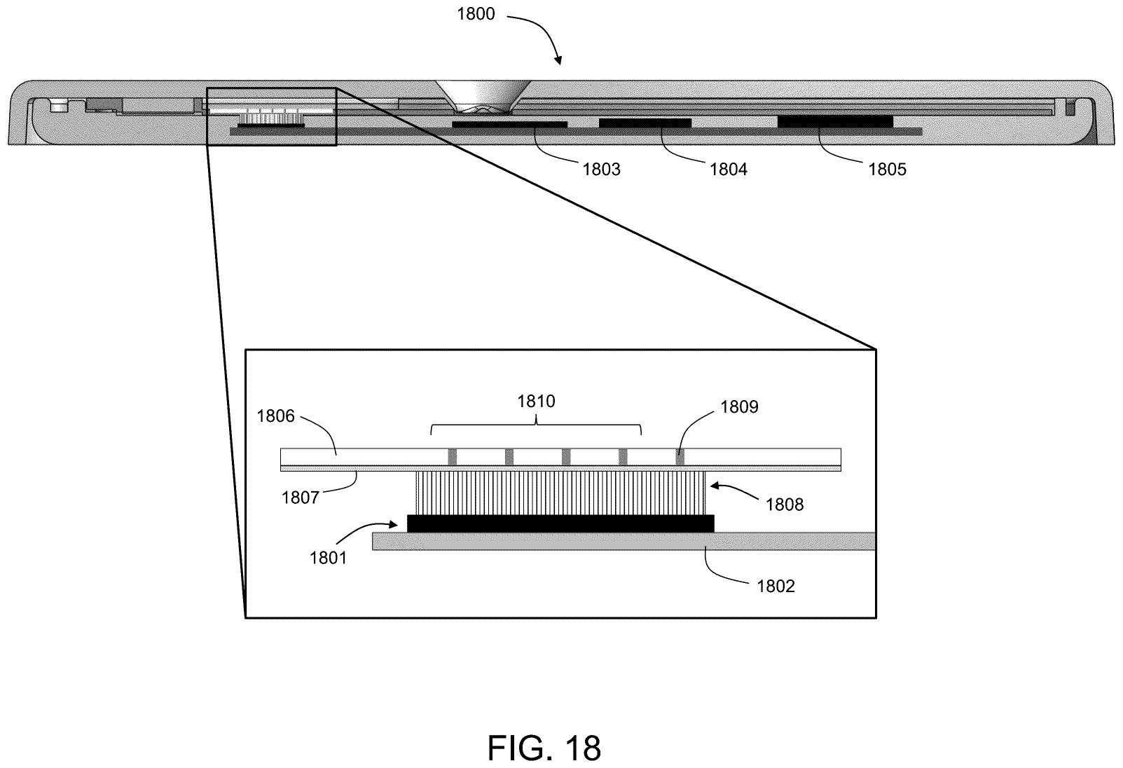

FIG. 18 depicts an initial embodiment of the integrated test cassette wherein a reader system is integrated into and encased within the CaDI cartridge 1800. In exemplary embodiments, the sensor 1801 is assembled to a printed circuit board (PCB) 1802 that comprises electronics including, for example, processors 1803, a wireless receiver/transmitter 1804, and a battery 1805. In this embodiment a light guide system is configured as a fiber optic plate 1808, or other light guide material (e.g., Fiber optic plate alternates, light guide plates, light guide films, fiber optic image bundles, fiber optic straight thrust, fiber optic image conduits, micro-structured optical films, and other light guide materials), bonded to the sensor array and to the nitrocellulose backing 1807, resulting in a lossless signal translation conduit, thereby enhancing signal translation and overall assay sensitivity. In the embodiment shown, the use of fiber optic plate 1808 reduces the size of the system by placing the subject and the sensor less than about 1-5 mm apart providing for integration of the sensor into the sample collection system.

In the embodiment illustrated in FIG. 18, a light guide material such as a fiber optic plate 1808 can be coupled to a nitrocellulose layer 1806 and backing layer 1807 and sensor 1801 via an optically transparent adhesive to minimize loss of photons from control 1809 and sample 1810 regions to sensor 1801. By removing any air gaps between the subject and sensor, signal loss that is present in traditional systems is effectively removed.

While not limiting embodiments of the present disclosure to the specific examples provided herein, in certain embodiments utilizing commercially available components, a sensor 1801 may be a Sony Pregius S IMX542 CMOS sensor and fiber optic plate 1808 may be a Schott fiber optic plate. In specific embodiments Norland NOA 61 optical adhesive may be used to bond fiber optic plate 1808 to sensor 1801 and Norland NOA 68 optical adhesive may be used to bond fiber optic plate 1808 to backing layer 1807. In certain embodiments, a PCB 1802 comprises a sensor, processor, memory, battery, and other necessary hardware incorporated into a single PCB that will be nested into a housing.

As shown in FIG. 18. a light guide may be placed between the sensor and the test strip within the cassette, bridging an air gap that may be present in some assemblies of the test strip and sensor. An air gap can result in the loss of the light signal from the test strip due to scattering, and/or refraction thereby reducing the light delivered to the sensor affecting the overall sensitivity of the assay. The gap generally being about 1 mm to 5 mm, including about 2 mm, or greater. In some embodiments the gap may be less than 1 mm including about 50 μm, 100 μm, 250 μm, 500 μm, and 750 μm. In certain embodiments of the test cassette, sensitivity of the assay could be improved via use of a light guide material such as a fiber optic plate, fiber optic cables, lenses, prisms, light pipes, waveguides, reflectors, diffusers, gels, oils, glues, adhesives or similar to bridge the gap between the sensor and the detection strip and minimize the loss of light signal from scattering and refraction in the air gap. Other embodiments may modify the layout of the components to eliminate the air gap such that the test strip is in direct contact with the sensor.

The dimensions of the test cassette should be considered flexible and are not constrained to a specific size range. Some embodiments of the test cassette may have a size that is constrained by the dimensions of the microfluidic system described in this disclosure. The size of the sensor is not constrained to a specific size range but could be selected to suit the dimensions of the cassette or the test strip. Commercial sensors selected for the construction of a proof-of-concept test would have a size large enough to cover the size of the test strip from which a chemiluminescent signal is generated. In other embodiments sensor sizes could include, but are not limited to, 3952 by 2320 pixels and a pixel size of 2 μm by 2 μm, such as a Sony IMX334 sensor, which is roughly 8 mm by 4.6 mm. Overall dimensions could range from about 25 mm or about 10 mm or about 5 mm or any integer between with a pixel size of about 2 μm or about 4 μm or about 6 μm or about 8 μm or about 10 μm, or any from about 2 μm or about 10 μm.

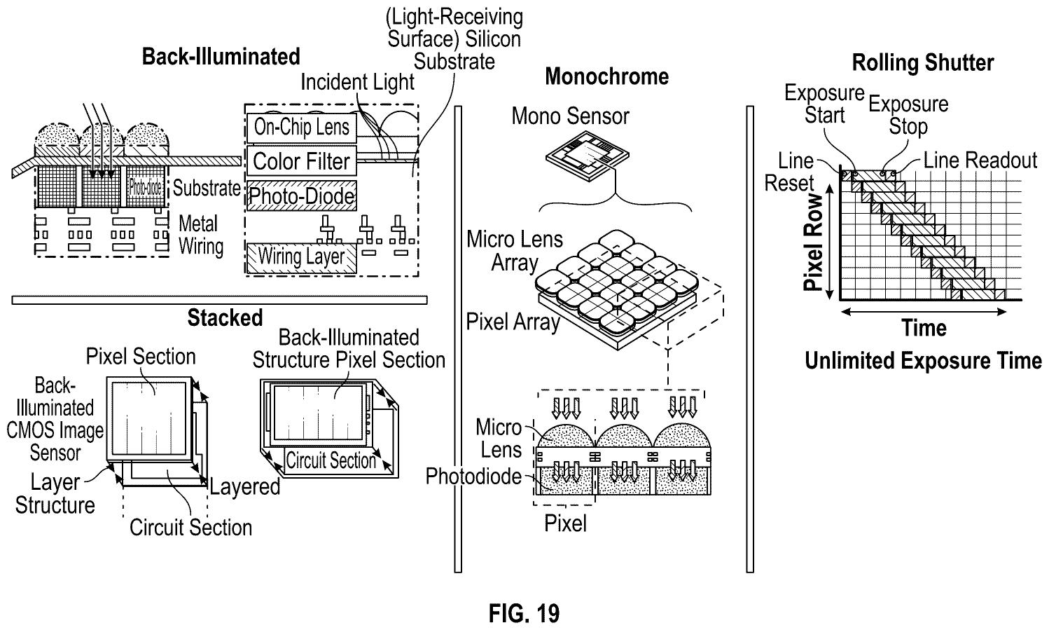

FIG. 19 illustrates aspects of sensor 1702 (see FIG. 17) and sensor 1801 (see FIG. 18) in certain exemplary embodiments. In particular embodiments, sensors 1702 and 1801 may be configured as back-thinned and back-illuminated monochrome sensors. In specific embodiments sensors 1702 and 1801 can be configured in stacked circuit with a pixel micro lens array. The features illustrated in FIG. 19 provide for high photon sensitivity with minimal background noise in the system. In specific embodiments, sensors 1702 and 1801 are also configured as rolling shutter sensors to provide high sensitivity while reducing manufacturing costs.

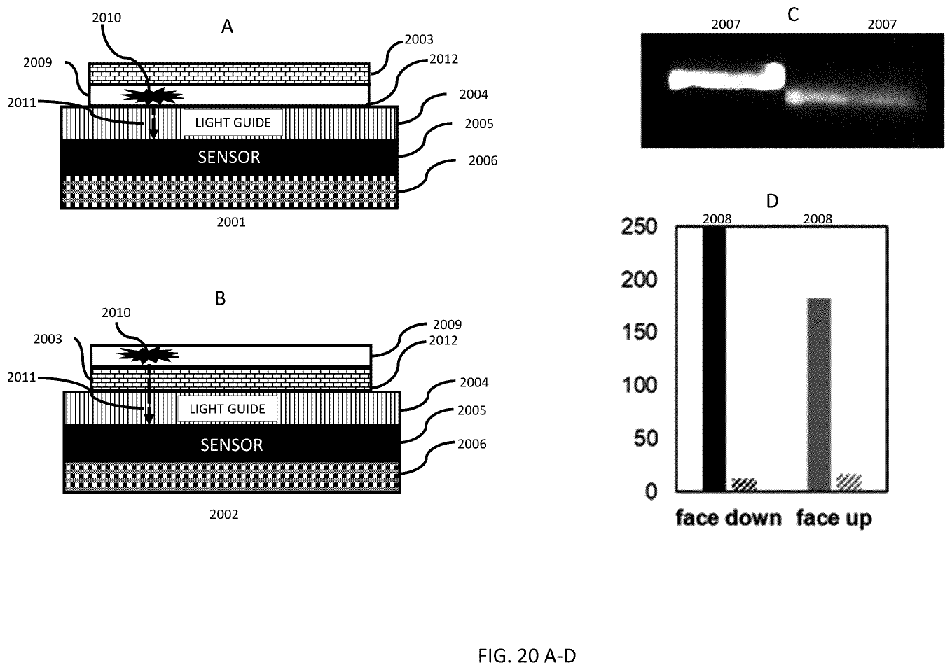

Referring to FIG. 20 A-D, a sensor in combination with a fiber optic plate shown as an embodiment light guide can be positioned relative to a nitrocellulose test strip in two distinct orientations here referred to as “face down” 2001 in panel 20 A or “face up” 2002 in Panel 20 B.

Referring to FIG. 20 A, the “face down” configuration 2001 involves positioning the sensor 2005 on a printed circuit board (PCB) 2006 directly above the test strip surface 2009 and the backing 2003, where the chemiluminescent signal 2010 is guided through a signal direction 2011 to the senor 2005 by the light guide fiber optic plate 2004 without having to transit the backing 2003. In one embodiment of the “face down” orientation 2001, the fiberoptic plate 2004 may be positioned above the test strip 2009 with a small air gap to prevent contact with the liquid content of the test strip 2009 introduced by the addition of a liquid sample to the cassette. In certain embodiments, the inclusion of an air gap could result in scattering or refraction of the chemiluminescent light signal 2010 away from the light guide fiber optic plate 2004 and subsequently the sensor 2005, reducing the sensitivity of the test cassette. In other embodiments, the fiber optic plate 2004 may be in contact with the liquid from the sample generating the signal 2010.

Referring to FIG. 20 B, in the “face up” orientation 2002, the sensor 2005 is positioned beneath the nitrocellulose test strip 2009 and backing 2003 wherein the chemiluminescent signal 2010 is produced. In some embodiments, the “face up” configuration may include the backing of the nitrocellulose test strip 2009 adhered to the fiber optic plate 2004 by an adhesive 2012, in some embodiments it may be a transparent adhesive, or double-sided adhesive, or other optically favorable adhesive. In other embodiments the test strip 2009 is adhered to the fiber optic plate by an adhesive without the presence of a backing to a nitrocellulose membrane or other test strip 2009. In yet another embodiment, the backing of the test strip 2009 may be held in contact with the fiber optic plate 2004 by the geometry of the cassette without the use of adhesive to bind the strip to the fiber optic plate.

The interaction of liquid between the light guide 2004 and test strip 209 may be adjusted by altering the hydrophobicity of the light guide, test strip membrane 2009 (e.g. nitrocellulose or other known in the art), surfactants, and backing. A hydrophilic light guide 2004 in contact with the test strip 2009 may alter the flow of the sample by acting as a secondary wicking material. In the case of a fiber-optic plate 2004 composed of glass, a hydrophobic coating may be used. In other embodiments, a fiber-optic plate 2004 composed of a plastic could provide innate hydrophobicity to the surface. The impact of contact between the light guide 2004 and test strip 2009 on liquid flow through the system would be considered in the orientation of the sensor 2005 and light guide assembly in some embodiments.

Exemplary embodiments of the “Face Up” and “Face Down” orientations of the test strip 2009 and sensor, with a fiber optic plate as a light guide 2004, have been evaluated experimentally as illustrated in FIG. 20 C and FIG. 20 D. In FIG. 20 C, a chemiluminescent image 2007 is depicted with “Face Down” on the left and “Face Up” on the right. The intensity of the image 2007 is quantified in a graph 2008 in FIG. 20 D. Both the “face up” and “face down” configurations can successfully capture chemiluminescent signal from the chemiluminescent reaction on the test strip 2009. In certain embodiments of the “face up” configuration, detection of the chemiluminescent signal may be impaired by the backing of the nitrocellulose test strip 2009, a double-sided adhesive (DSA) 2012, or other securing materials.

The nitrocellulose backing 2003 and a DSA 2012 are generally composed of plastics such as polyester (PET), Polyethylene(PE), polyvinyl Chloride (PVC), Polycarbonate (PC), and polypropylene(PP). In some configurations, the adhesive may be applied to one side of the plastic material and the nitrocellulose membrane coated onto the opposite forming a single component with an adhesive side and a nitrocellulose side, eliminating the need for a second double sided adhesive tape. A backing for the nitrocellulose test strip 2003 is generally included to improve the manufacturability of the test strip, as nitrocellulose membranes without a backing is fragile and prone to puncture, tearing, or other damage, requiring careful handling. Generally, nitrocellulose (or other membrane) test strips 2009 are coated with antibodies on one surface opposite the backing of the strip. In some specific embodiments, the backing of the nitrocellulose test strip 2009 may be excluded, and the nitrocellulose 2009 could be coated with antibodies on both sides. In other embodiments, the nitrocellulose 2009 can be adhered directly to a light guide material 2004. In another embodiment the nitrocellulose can be adhered directly to the sensor 2005.

Exemplary embodiments of a sensor and a light guide shown in FIG. 18 and FIG. 20A-D utilize a fiber optic plate as a light guide. A fiber optic plate is composed of a matrix of individual fiber optic cable elements, which allows for lossless transfer of incoming photons from the test strip to the sensor. In some embodiments, a lens may be utilized as a light guide. In some embodiments with a lens, the lens may result in a loss of sensitivity due to scattering and refraction of light or increase the size of the sensor and/or light guide assembly to accommodate the focal length and working distance of the lens.

In some configurations, the dimensions of the fiber optic plate may be made to exactly match the size of the sensor. In other aspects, the fiber optic plate may be larger or smaller than the sensor to which it is assembled. In some specific configurations, the fiber optic plate may be manufactured to match the pixel density of the paired sensor. In some specific embodiments, the fiber optic plate may be manufactured in combination with the test strip resulting in a fiber optic plate serving as a backing to the test strip. Incorporating a fiber optic plate as a backing to the strip could simplify the assembly process of the cassette by combining two separate components into one.

Referring back to FIG. 19, commercially available back-illuminated sensors can demonstrate improved sensitivity with larger pixel sizes, in part because the overall area taken by circuitry is reduced in larger pixels with traditional back-illuminated sensors. The ratio of the light sensitive area of a pixel versus the total area of a pixel is referred to as the fill-factor, wherein a pixel with a larger fill-factor has more area of the pixel dedicated to light sensitive photodiodes. The fill-factor for larger pixels, including pixels above about 4 micron, is generally high in comparison to smaller pixels, resulting in an improved sensitivity of larger pixels to photons. As a result, sensors with high resolution, and low pixel sizes, may be less sensitive to light as a result of a lower fill factor.

Stacked back-illuminated pixel designs do not have pixel area taken up by circuitry, thereby having a larger fill-factor, but the cost and manufacturing complexity of stacked sensors is significant which may limit the viability of stacked back-illuminated sensor designs in developing a low-cost test cassette.

Commercial sensors considered in the construction of exemplary embodiments of the test cassette were sourced from camera equipment spanning a range of costs, reflecting the size and sensitivity of the sensor component. Two sensors tested in exemplary embodiments of the test cassette were color sensors. The oxidation of luminol in the chemiluminescent reaction produces visible light in the near blue range, typically about 425 nm. Because CMOS sensors cannot differentiate between the wavelengths of light detected, the color sensor uses a color filter pattern over the pixel array to capture light information from the image. As a result, when only one color is present, many pixels in the sensor are unused and unable to detect incident light as the colored light is filtered out by the color filters and does not reach the light detecting photodiode of the CMOS sensor pixel. Color filters, therefore, reduce the sensitivity of the CMOS sensor when only one color is present.

In some high-sensitivity embodiment applications, the particular color of the light detected may not be of importance and a monochrome sensor may be preferred. A monochrome sensor omits the color filter, such that every pixel can detect incident photons but cannot correlate the photons with a color. As a result, monochrome sensors are more sensitive in low-light conditions and are preferred in certain applications. A monochrome sensor used in some exemplary proof of concept embodiments of the cassette was a sensor commonly used in security cameras. Evaluations of such embodiments demonstrated that a monochrome sensor would be preferred in a test cassette to improve the sensitivity of the sensor to detect photons from the chemiluminescent reaction.

Commercially available monochrome sensors currently available are generally developed for high-speed applications, and are more expensive than color sensors that may be used in a camera. Commercially available camera sensors are offered in various size ranges, including but not limited to 3.6 mm×2.7 mm, 6.4 mm×4.8 mm, 8.8 mm×6.6 mm, and others. Specific limits to the sensor size are not disclosed herein, and the size of the sensor may be selected based on the specific embodiment. From testing results of some exemplary embodiments, the highest-cost monochrome sensor outperformed others in detecting the chemiluminescent signal from the assay.

In some embodiments of the apparatus, the commercially available sensor could generate significant amounts of heat. High temperatures could impact the performance of the chemiluminescent assay. In such embodiments, cooling system such as a heat sink or Peltier plate or combination thereof, or other heat extraction system may be incorporated into the test cassette to remove heat from the commercial sensor.

As mentioned, commercially available sensors may include features that could impact the construction of the cassette. For instance, some sensors are developed for high-speed video capture and may generate large amounts of heat. Other commercially available camera sensors may be developed for high resolution image capture, or color sensing, and thereby have reduced sensitivity in the context of a chemiluminescent assay. In some embodiments the test cassette may utilize a commercial sensor with certain features not well suited to the chemiluminescent assay contained in the test cassette. In other preferred embodiments, a custom sensor may be developed with unique characteristics well suited for the chemiluminescent assay.

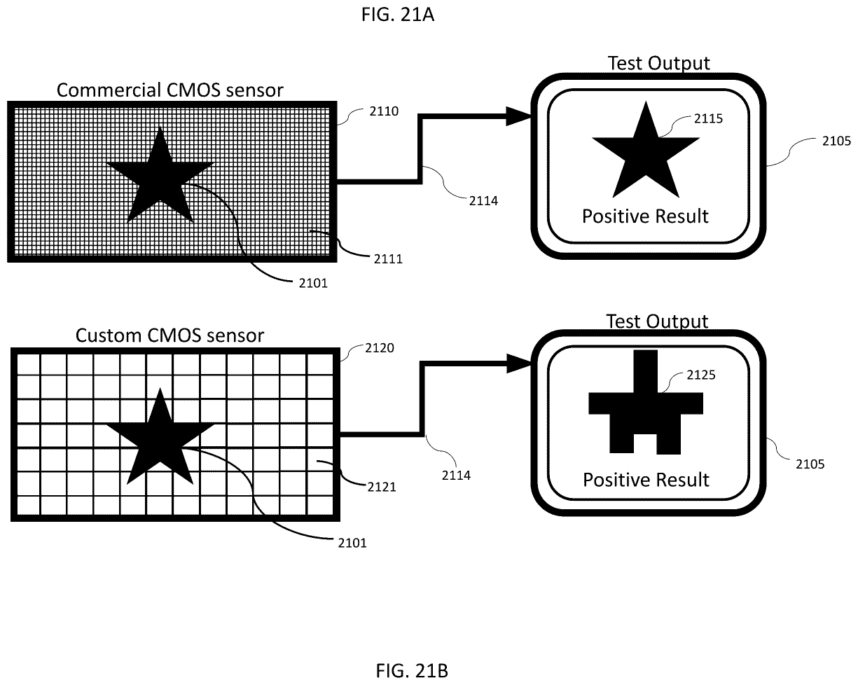

Depicted in FIG. 21 is a comparison of a commercial sensor in the top panel and custom sensor in the bottom panel that may be utilized in some embodiments of the test cassette. Certain embodiments may use a commercially available sensor shown, developed for high resolution imaging 2110. In such embodiments, the sensor captures light 2101 with an array of dense pixels 2111 and produces a signal. The signal is then exported, interpreted by software and a processor 2114, and a result is displayed 2105 indicating whether the test is positive or negative for the specific biomarker. A high pixel density could allow for a more accurate reproduction of the image 2115 at high resolution if necessary.

In many configurations an accurate high-resolution reproduction of chemiluminescence is not necessary in determining whether a sample is positive or negative for the biomarker of interest.

Rather, a high-sensitivity custom sensor with a low pixel density and corresponding lower resolution may be selected instead of a high pixel density and high resolution. In embodiments a high pixel density is considered to be composed of pixels less than about 3.5 micron in size, included pixel sizes of about 1 micron to about 1.7 micron and about 1 micron to about 2.2 micron, or about 2.0 micron to about 3.5 micron. Some embodiments may instead use a custom sensor 2120 design with a low pixel density 2121, and larger pixels, wherein low pixel density describes pixel sizes ranging from about 4 micron to about 14 micron, including about 4.1, 4.2, 4.3, 4.4, 4.5, 4.6, 4.7, 4.8, and 4.9 micron, or about 5.0, 5.1, 5.2, 5.3, 5.4, 5.5, 0.56, 5.7, 5.8, 5.9, 6.0 6.1, 6.2, 6.3, 6.4, 6.5, 6.6, 6.7, 6.8, 6.9, or 7.0 microns, about 10 micron, and about 11 micron.

Embodiments of the test cassette using a custom monochrome sensor with a low pixel density could detect the chemiluminescent image 2101, interpret the signal via a processor and software 2114, and display 2105 a highly accurate result 2125 without the higher image resolution of a commercial sensor with a high pixel density. The use of such a custom sensor with a lower pixel density and larger pixel size in comparison to common, high-resolution, commercial sensors could reduce the overall manufacturing costs of the cassette while improving the sensitivity of the assay. While monochrome CMOS sensors are commonly used in such high-sensitivity low-light applications, this does not exclude the use of other sensor types for inclusion into a cassette. Alternative sensor technologies include Charge-Coupled Device sensors (CCD), Single-Photon Avalanche Diode sensors (SPAD), photoelectric sensors, or other sensor suitable technologies not yet developed or disclosed.

The precise size of the custom sensor should be considered flexible. In specific embodiments, the sensor may precisely match the size of the test strip. In certain embodiments, a sensor size of about 3 mm by about 7 mm may match the size of the corresponding test strip.

In FIG. 21 the test result readout from the cassette is represented as a digital display 2105 displaying the image captured by sensor 2115 and 2125. This is but one basic embodiment of the readout that may be delivered to the user when using the cassette. In some embodiments, the readout of the cassette may be delivered wirelessly to an external device such as a user's phone or computer wherein the readout could be shown on a display of the external device. In certain embodiments wherein the cassette is connected to an external device by a cable, such as a USB cable for power and signal transmission, the readout may be presented on the display of the external device. In some embodiments, the readout may only report whether the sample was positive or negative for a target analyte contained in the sample. In other embodiments, quantitative data may be determined and presented, such as the signal intensity in specific regions of the detection strip. In yet another embodiment, the readout may be presented on a display on the cassette itself or otherwise on the cassette itself.

In certain embodiments, the signal could be presented on a display situated on the outer surface of the cassette. In some aspects, a display may be produced as a flexible display. In other embodiments, LEDs may be used to report the result by switching the LED on or off. In yet other embodiments, the signal may be presented via speaker emitting sounds depending on whether the sample was positive or negative. The methods of reporting a result described in this disclosure should not be considered limiting, and other methods of reporting a result may be considered in the design and manufacture of the cassette.

The result of the test is derived through the analysis of the chemiluminescent signal captured by the sensor. In certain embodiments, an on-board processor such as a CPU, or custom state machine may be incorporated into the sensor chip, within the cassette is responsible for this interpretation. Alternatively, in certain embodiments, the sensor signal interpretation is delegated to an external device, such as a phone or computer, connected via USB or wirelessly to the cassette. In such configurations, an on-board processor such as a CPU, or custom state machine integrated into the sensor chip, in the cassette may be excluded from processing and interpreting of the output signal from the sensor if it is performed using software and a processor or processors of a connected device. Such embodiments may require random access memory (RAM) to store the sensor signal output for transmission.

Software would be utilized to interpret the output signal from the sensor and determine whether the signal corresponds to the presence or absence of the biomarker being tested, as well as the presence or absence of chemiluminescent emission from the control region of the test strip. Such software may utilize machine vision task for determining positive and negative signals from sample. Additional software embodiments may be included as necessary to operate other components of the cassette, such as a LED drivers for LED components or Bluetooth drivers for a Bluetooth transmitter.

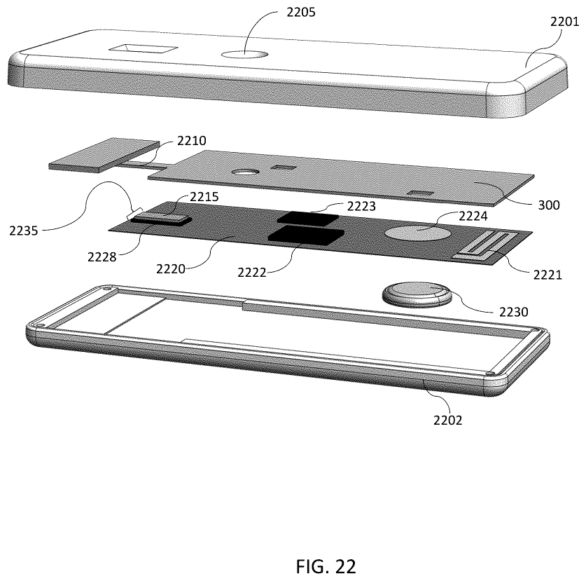

FIG. 22 depicts one exemplary embodiment of the cassette which would include a first component of the cassette housing 2201, a power source such as a battery to power the cassette 2230, a custom sensor chip 2228, printed circuit board (PCB) 2220 including a wireless transmitter 2221 and battery terminal 2224, a processor such as a CPU 2223, other components 2222 such as a switch or display, a light guide (fiber optic plate) as previously described 2215, a microfluidic chemiluminescent system as previously described 300 with a test strip 2210, and second component of the cassette casing 2202 including sample inlet 2205.

FIG. 22 depicts an embodiment of the cassette with just one internal compartment for all components of the cassette. In some embodiments, this single compartment may encompass a combination of a microfluidic system 300, sensor assembly 2235, batteries 2230 or other power elements, and circuitry elements such as a processor such as a CPU 2222 and wireless transmitter 2221. The separate components of the cassette housing 2201 and 2202 may be combined via conventional methods, including but not limited to press fit, snap fit, mechanical fastening, welding, adhesives, or other methods familiar to those skilled in the art. In some embodiments, the cassette casing, including the lid, or base may be composed of common thermoplastics appropriate to the application such as polypropylene (PP), high-density polyethylene (HDPE), Acrylonitrile Butadiene Styrene (ABS), or polycarbonate (PC), or other polymers considered appropriate for the application. Other embodiments of the cassette housing may instead be composed of materials other than thermoplastics, such as photocured resins.

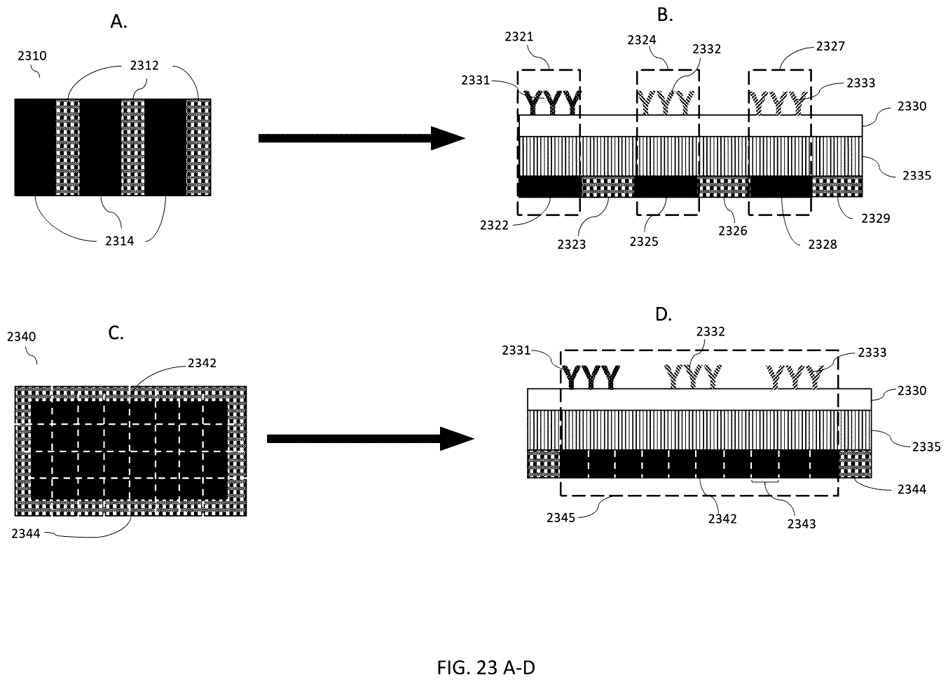

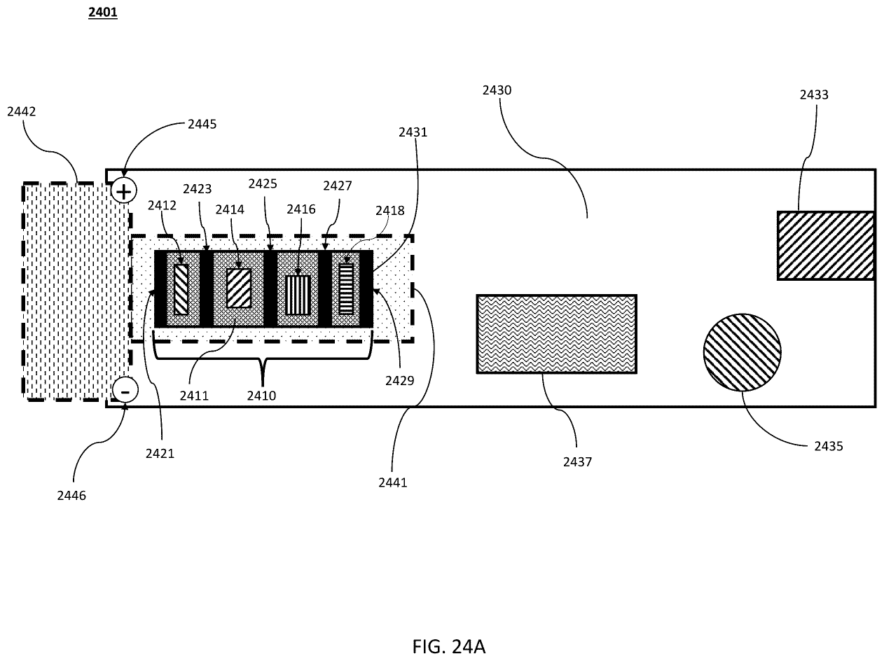

Referring now to FIG. 23 A-D, a custom sensor element embodiment may take different forms to meet constraints in price and manufacturability. In certain embodiments depicted in FIG. 23 A, 2310, embodiments of the sensor may be a series of pixel islands 2314 with circuitry 2314 positioned between the pixel islands. In such embodiments, the pixel islands can be separated into regions that align with the detection regions of the test strip 2330 with a light guide embodiment fiber optic plate 2335 between the sensor and the test strip 2330. For instance, referring to FIG. 23 B, embodiment 3 pixel islands are present in the custom sensor 2310 comprising of: a control pixel island 2321 composed of control antibodies 2331, the control sensor pixels 2322, and the adjacent circuitry 2323, a first test pixel island 2324 composed of first test antibodies 2332, first test sensor pixels 2325, and adjacent circuitry 2326, and a second test pixel island 2327, composed of second test antibodies 2333, second test sensor pixels 2328, and adjacent circuitry 2329. In other embodiments of the custom sensor element 2340 manufactured for this application, a single pixel region 2342 surrounded by circuitry 2344 wherein a single sensor unit is formed 2345 comprised in this case of a control antibody 2331, test 1 antibody, 2332, test 2 antibodies, and a pixel sensor region 2342 positioned below the antibody regions of the test strip. In some embodiments, the sensor may utilize larger pixel sizes for increased sensitivity and reduces manufacturing costs.

In embodiment custom sensor chips, specific combinations of circuitry may be utilized to maximize the sensitivity of the pixels. For instance, referring again to FIGS. 23 A and 23 B, in some embodiments the antibody deposition on the test strip may be matched to pixel islands on the sensor. In such embodiments, the antibodies may be deposited on the nitrocellulose membrane of a test strip in a width equal to the combined width of about 2 pixels. In exemplary embodiments, the photodiodes of the pixels may be aligned with the antibodies and the pixel circuitry is oriented around the perimeter of the pixels to increase the fill-factor and sensitivity of the pixels. In this manner, the detection area of the pixel is maximized by removing the need to place circuitry within the pixel area itself. In specific embodiments, the use of pixel islands would not require the use of a stacked sensor design and the associated costs. By contrast, FIGS. 23 C and 23 D demonstrate an embodiment with a sensor array design wherein the pixels at the center of the sensor have circuitry that may consume some space of the pixel while the pixels at the perimeter of the sensor could place circuitry adjacent to the photodiode region. In some embodiments, a combination of pixel designs may be utilized, for instance pixels of the interior of the pixel array utilize the stacked pixel design placing the circuitry beneath the light sensitive photodiode.

Referring again to FIG. 23 A-D, the configurations of the sensor presented should not be considered limiting. Depending on the application embodiment custom sensor chips may be utilize traditional back-illuminated pixel designs, with circuitry placed on the pixels, in combination with pixel islands with circuitry arrayed at the perimeter of the pixel islands. In some embodiments, this may involve a wide number of pixel islands arranged to suit the application and may span a range including 1, 2, 3, 4, 5, 6, 7, 8, 9, or 10 pixel islands on the sensor or any number in the range of 10-100 islands, or any number greater than 100. The shape of the pixel islands should also be considered flexible. In some embodiments wherein antibodies are deposited in a line on the nitrocellulose membrane of a test strip, the pixel islands may be rectangular. In other embodiments wherein the antibodies are deposited as dots, the pixel islands may be square or circular. In yet other embodiments, the pixel islands may be of some other shape. The deviation of the custom sensors disclosed herein from traditional CMOS camera sensors allows for flexibility not currently available in area CMOS sensors designed to capture images with high resolution or sensitivity. In the applications disclosed herein a single uniform image of the test strip is unnecessary in some embodiments to provide a result and a unique arrangement of the pixels on the sensor chip may be appropriate. In other embodiments, traditional area sensor designs composed of a single uninterrupted array of pixels containing individual circuitry elements may be utilized.

Linear CMOS sensors of low pixel width are currently used in scanning image capture wherein the sensor is swept over the area of interest to generate the image one pixel row at a time. The pixel islands disclosed herein for embodiments differ from these linear CMOS sensors in construction and application. The pixel island construction in embodiments of the present disclosure would be formed in a fixed pattern on the sensor. In some embodiments, the regions of interest on the test strip would be aligned with the fixed pattern during the manufacturing process. In exemplary embodiments, the fixed sensing regions, composed of pixel islands, of the chip would detect chemiluminescence from the corresponding regions of the test strip that are aligned with the sensing regions. The fixed pixel island pattern is considered flexible. In some embodiments, discrete pixel islands may not be present and instead one continuous pixel pattern is utilized. In specific embodiments, the pixel pattern may be linear. In other embodiments, the pixel pattern may be a different shape, for instance an S-shape, a curved shape, or a combination of different shapes. In some embodiments, the pixel pattern or pixel islands may be only 1 pixel in width with circuitry fabricated along the side of the light sensing photodiode. In other embodiments, the pixel pattern or pixel islands may be 2 pixels in width, 3 pixels in width, 4 pixels in width, 5 pixels in width, 6 pixels in width, 7 pixels in width, 8 pixels in width, 9 pixels in width, 10 pixels in width, and so on, or greater.

In specific embodiments of the pixel islands, the islands may be square in the form of a 2×2, 3×3, 4×4, 5×5, 6×6, 7×7, 8×8, 9×9, or 10×10 array of pixels. Alternative embodiments of the pixel islands may be of a size to match the size of the size of the antibody depositions on the test strip. The spacing of pixel islands should be considered flexible based on the spacing of the detection areas of the test strips. Pixel islands may be spaced by 1 pixel width, 2 pixel widths, 3 pixel widths, 4 pixel widths, 5 pixel widths, 6 pixel widths, 7 pixel widths, 8 pixel widths, 9 pixel widths, 10 pixel widths or any other spacing greater than 10 pixel widths. In certain embodiments, the pixel islands may be larger than the corresponding detection area of the test strip to account for shifts in alignment between the test strip and the sensor that may arise during manufacture.

Within certain manufacturing methods, antibodies may be deposited onto the test strip in widths ranging from about 0.4 mm to about 1 mm, or 400-1000 micrometers, in some embodiments. In such embodiments, pixel islands may be expanded to meet the limitations of the manufacturing method. In certain embodiments, pixel islands may range from 100, 200, 300, 400, 500, or 600 pixels in width. The width of the pixel islands is defined by the width of the individual sensor pixels and the width of the detection areas of the test strip. The dimensions of the pixel islands may also be selected based on the dimensions of the test strip. In some embodiments, a test strip with a width of about 3 mm may have pixel islands with a width of about 500 pixels, about 600 pixels, about 700 pixels, about 800 pixels, or some dimension between about 100 pixels and about 1,000 pixels.

The size of the sensor, and the corresponding dimensions of the silicon wafer from which the sensor chip is fabricated may further be defined by the dimensions of the test strip of the chemiluminescent assay. In specific embodiments, the sensor and silicon wafer may be of about 3 mm×about 7 mm in size to match a about 3 mm×about 7 mm test strip. Other sizes may similarly be considered with widths ranging from about 1 mm, about 2 mm, about 3 mm, about 4 mm, about 5 mm or some dimension between these values and lengths ranging from about 3 mm, about 4 mm, about 5 mm, about 6 mm, about 7 mm, about 8 mm, about 9 mm, about 10 mm, about 11 mm, or about 12 mm, or some dimension between these values. The dimensions of the test strip and corresponding sensor/silicon wafer should be considered flexible and not necessarily constrained to the dimensions disclosed herein.

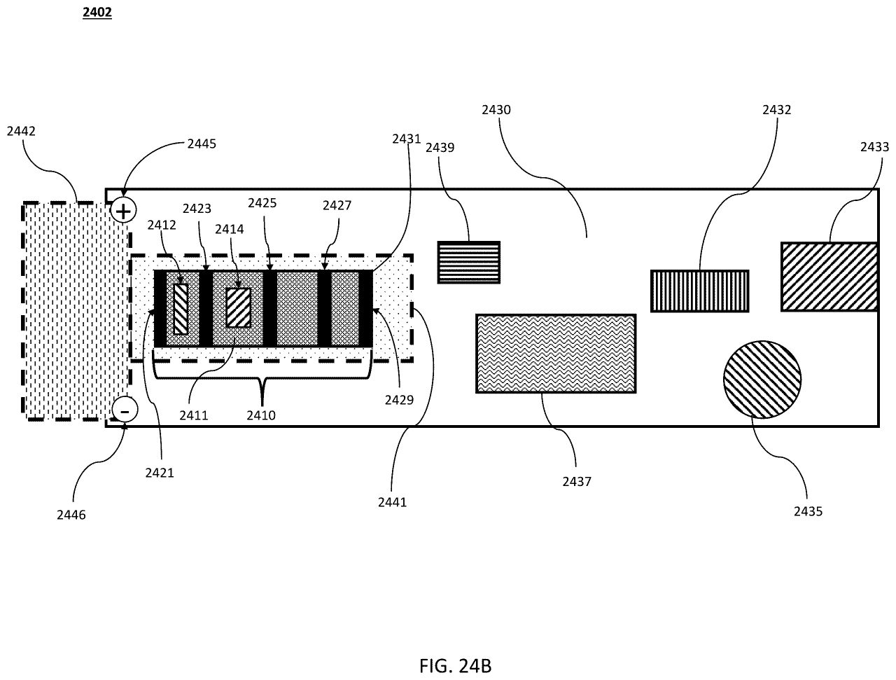

Referring now to FIG. 24 A and FIG. 24 B, illustrate exemplary embodiments of the control system 2401, 2402, that could be incorporated into the test cassette. Embodiments of the custom sensor chip 2410 in FIGS. 24 A and 24 B are positioned beneath the test strip 2441 where the chemiluminescent signal is generated. A light guide 2431 may be placed between the test strip 2441 and the custom sensor chip 2410. In the exemplary embodiment, the custom sensor chip 2410 is a CMOS sensor chip and is fabricated from a bed of silicon 2411 and may contain five pixel islands 2421, 2423, 2425, 2427, and 2429 in the shape of a rectangular stripe. As described previously, the size, shape, number, and orientation of the pixel islands on a custom CMOS sensor chip should be considered flexible and represent additional embodiments and are not limited to the exemplary embodiment described by FIG. 24 A and FIG. 24 B.

For embodiments, the shutter time for the CMOS sensor may be selected to maximize signal sensitivity towards the chemiluminescent reaction. The shutter time refers to the period of time over which a sensor is exposed to photons before offloading the sensor signal. The shutter time for the CMOS sensor is a characteristic of the sensor that can be adjusted in the design of a custom sensor. A long shutter time allows for more photons to be detected by the sensor, increasing the sensitivity of the sensor. In the low light conditions of the cassette, a shutter time of 16 milliseconds may be selected in some embodiments. In other embodiments a shutter time of 50, 100, 200, 300, 400, 500, 600, 700, 800, or 900 milliseconds may be utilized to tune the assay sensitivity. When the analyte of interest is generally at low concentrations, longer shutter times of 1, 2, 3, 4, 5, 6, 7, 8, 9, or 10 seconds may be utilized. In other embodiments shutter times between 10 and 60 seconds may be utilized, including but not limited to 20, 30, 40, and 50 seconds. In some specific embodiments, shutter times over 1 minute may be selected when developing the custom CMOS sensor.

The custom sensor chip 2410 allows for the placement of necessary peripheral circuitry between the individual pixel islands. Traditional area CMOS sensors have a matrix of pixels, and peripheral circuitry has to be placed at the perimeter of the sensor and increase the overall size of the sensor chip. Incorporating peripheral circuitry between pixel islands helps to reduce the overall footprint of the chip and maximize the efficient usage of the silicon bed when fabricating the sensor chip, which reduces the overall cost. In FIG. 24 A power management circuitry element 2412 is placed between pixel islands 2421 and 2423, communications circuitry element 2414 is positioned between pixel islands 2423 and 2425, a processor custom state machine circuitry element 2416 is positioned between pixel islands 2425 and 2427, and wireless transmitter circuitry element 2418 is positioned between pixel islands 2427 and 2429.

The circuitry elements, 2412, 2414, 2416, and 2418 as well as additional circuitry may represent a wide variety of embodiments with functions or components on the chip and can even incorporate functions that might be covered by external components, such as a processor, CPU, custom state machine, or even wireless transmitter. For instance, in some embodiments circuitry element 2412 may serve as the power management circuitry of the chip when connected to the power source 2435 by a printed circuit board (PCB) or a flexible printed circuit board (FPCB) 2430. In other embodiments circuitry element 2414 may serve as communication circuits for the chip to communicate with other components such as a processor or CPU 2432, Bluetooth chip 2439, or connective port 2433. In yet other embodiments, the circuitry element 2416 may be a custom state machine to process the sensor signals and generate a reading without the need for an external processor or CPU 2432. In some embodiments, circuitry element 2418 may serve as a wireless transmitter or Bluetooth transmitter, thereby eliminating the need for an external Bluetooth chip 2439. In other embodiments, additional circuitry components such as a summer may be included. The precise placement and function of the circuitry elements on the custom CMOS sensor chip should be considered flexible and are not constrained to the specific orientation and function disclosed herein. Other embodiments may add or remove circuitry elements from the custom CMOS sensor chip to suit changing needs of the test cassette. Similarly, the number of pixel islands may be adjusted to meet the application.

Referring now to FIG. 24 B, the custom sensor chip 2410 is affixed to a PCB or FPCB 2430 onto which other components necessary for the function of the control system are affixed. In some embodiments, certain functions such as processing or wireless communication may be facilitated by separate components integrated onto the PCB or FPCB. For instance, in some embodiments a processor or CPU 2432 external from the sensor chip 2410 may be included to process the signal from the sensor. In other embodiments wherein wireless connectivity is a desired feature, a wireless transmitter 2439 may be included. In some embodiments the wireless transmitter 2439 may be a Bluetooth transmitter In certain embodiments, some necessary circuitry may still be present on the sensor chip, such as power management circuitry 2412 or communications circuitry 2414 to package and transmit the sensor signals to other components external from the sensor chip.

Power delivery to the cassette may be achieved through a power source 2435. In some embodiments, particularly those without a port for wired connectivity, the cassette is powered by a battery. The battery may generally be a coin battery in some embodiments, but could also be another type of battery. Other embodiments of the cassette may be powered by an external power source such as a phone, tablet, or computer. In such embodiments, the cassette may not need to contain its own source of power such as a battery. In certain embodiments, an external power source may be connected to the cassette via wire and a connection port 2433 on the cassette. In specific embodiments the connection port 2433 may be a USB connection port. In some embodiments, a different type of wired connection port may be utilized. In certain embodiments, data may be transferred to an external device, such as a phone, computer, or tablet, by a wired connection to the connection port 2433 instead of a wireless transmitter 2439 or 2418.

In some embodiments, certain miscellaneous components 2437 may be included on the board to suit the application. For instance, in some embodiments, component 2437 may be an activation switch or button to turn on the device or initiate reading of the sensor signal. In other embodiments, component 2437 may be a display component such as a display, LED, speaker, or similar or different element known in the art.

Starting the cassette for operation and addition of a sample may be performed in a number of ways, or via a series of steps. In certain embodiments, the user may pull on a tab to remove a barrier that prevented contact between the battery and the battery terminal. In other embodiments, USB or other physical connective port is designed into the cassette such that power will be supplied to the cassette via connection to an external device such as a computer or phone or tablet. In some embodiments, the user may press a button or slide a switch to power on the cassette and initiate reading of the test strip. A solution or liquid sensing switch 2445, 2446 may also be used in some embodiments. Switches may generally be used in some embodiments to reduce power consumption from the power source 2435 until necessary thereby extending the shelf life of the test cassette, or reduce the power requirements for the power source 2435.

There is a generally delay in LFA between when a sample is added to the inlet of the cassette and when chemiluminescence is detectable by the sensor 2410. In some embodiments, the user may press a button when depositing a sample, which would start a clock to only initiate reading by the sensor 2410 after a pre-set period of time. In another embodiment, a solution or liquid sensing switch may be incorporated to only activate reading once the liquid sample has passed through the test strip. In the exemplary embodiment in FIG. 24 A and FIG. 24 B, a solution or liquid 2445, 2446 switch has positive terminal 2445 and negative terminal 2446 are positioned at opposite ends of the wicking reservoir of the microfluidic system 2442. The wicking material 2442 is generally nonconductive so a circuit cannot be completed between the positive 2445 and negative 2446 terminals. Once liquid from the sample reaches the wicking material, after flowing through the microfluidic system, the liquid can complete a circuit between the positive 2445 and negative 2446 terminals. In this embodiment, the time delay between addition of a sample, and reading by the sensor, is facilitated by the liquid sensing switch at the wicking reservoir 2442. Other embodiments may adjust the liquid sensing switch and select different areas to place the positive 2445 and negative 2446 terminals.

In other embodiments, a temperature sensor may be included to determine whether an accurate reading can be produced. In such embodiments, the user may be alerted that the ambient temperature, such as high temperature regions, of the cassette could impact the accuracy of the assay.

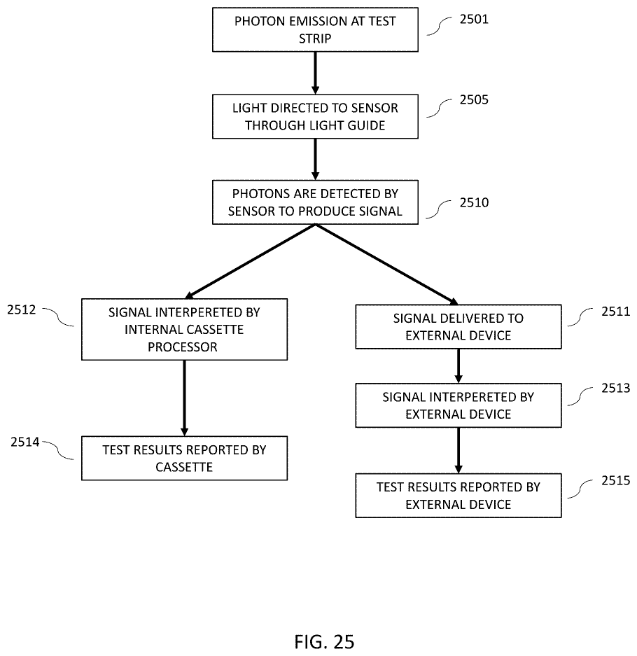

Referring now to FIG. 25 presents the function of a cassette following delivery of the sample to the cassette, and completion of the reaction sequence facilitated by the microfluidic system described previously in this disclosure. Photons are emitted from the detection area of the test strip by a chemiluminescent reaction 2501. Photons from the detection area of the test strip are directed through a light guide, such as a fiber optic plate, to the sensor 2505. Photons are detected by the sensor to produce a signal 2510. In some embodiments, the signal from the sensor is delivered to an external device either wirelessly or by wired connection 2511, the signal is interpreted by software on the external device 2513, and finally the test result is reported to the user on the external device 2515. In other embodiments that do not involve an external device, the signal from the sensor is interpreted by software on an internal processer, such as a processor, custom state machine, or CPU, in the cassette 2512, and the test result is reported by the cassette in a method such as an LED light, a display, or a sound 2514. In certain embodiments, the signal could be interpreted by an internal cassette processor, and then delivered wirelessly to an external device to display the result.

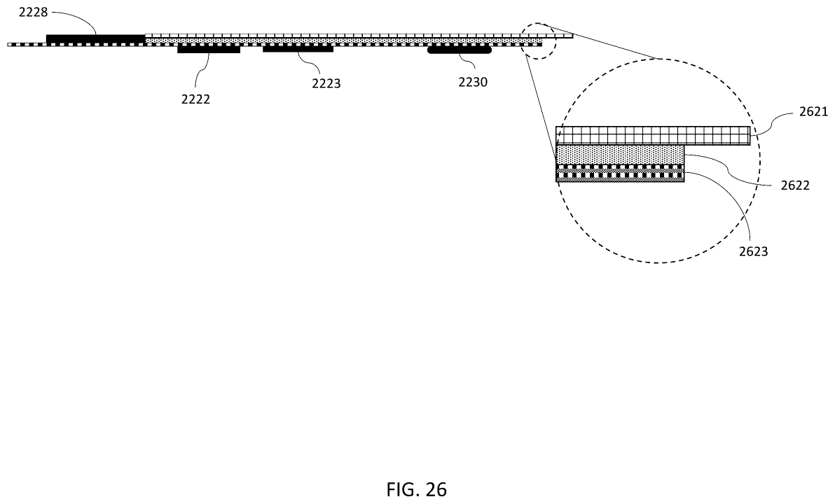

Referring now to FIG. 26, the PCB of the cassette may be produced as a FPCB 2623 in embodiments. The incorporation of a FPCB design could facilitate a roll-to-roll manufacturing process as part of the manufacture for the sensor component. In such a process, the FPCB 2623 could be efficiently combined with an adhesive tape 2622 and a liner 2621 in a high-throughput fashion. The addition of the sensor chip 2228, processor or CPU 2223, other circuit elements 2222, and power source such as a battery 2230 (or USB connection) together would form a flexible, adhesive component that could be adhered to a surface. In some configurations of the cassette, the adhesive FPCB sensor unit could be adhered to the interior of the cassette by removing the liner 2621 such that the adhesive 2622 is exposed and able to contact the interior surface of the cassette. The combination of a FPCB 2623, adhesive 2622, and sensor 2228 should not be considered specific to the manufacture of the cassette. In some embodiments, the manufacture of the cassette may be further simplified by combining the sensor 2228, processor or CPU 2223, wireless connectivity 2221 or other components 2222 into a single chip manufacture and therefore a single component. Combination of these components into a single chip could reduce assembly time, costs, and complexity and represent additional embodiments.

The flexible chemiluminescent sensor could be adapted to meet new needs wherein a disposable, adhesive sensor can be used to perform chemiluminescent detection without the need for a secondary reader.

Additional embodiments represent modifications to the sensor pixel pattern during the manufacturing process, such as the pixel islands previously described, allow for diverse applications in the field.

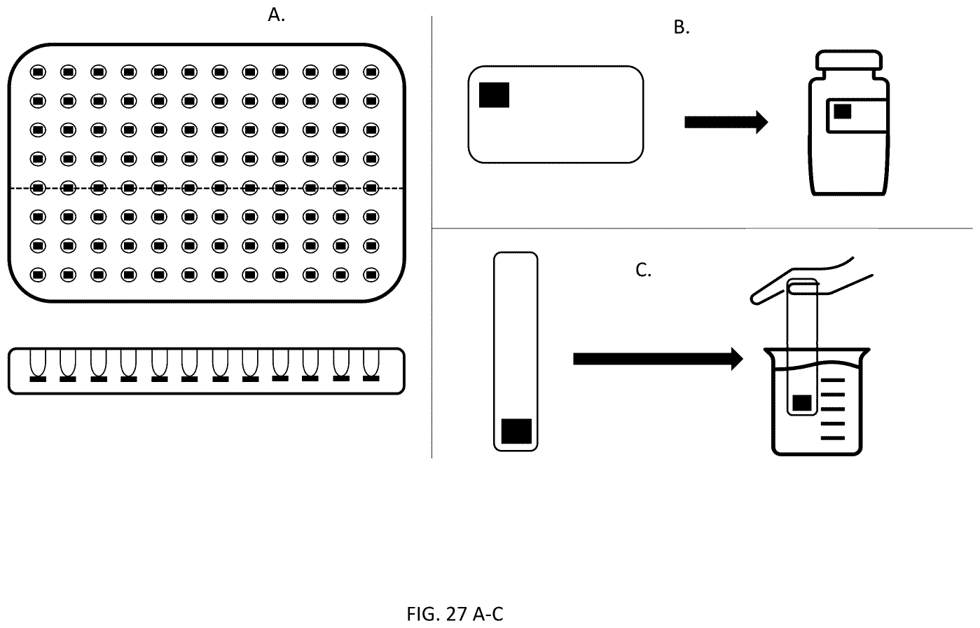

Referring to FIG. 27 A, panel shows embodiments where a pattern of 96 individual pixel islands are arrayed into a single chip to form a base onto which a sample reaction plate can be placed. The sensor can evaluate the chemiluminescence emitted from a chemiluminescent reaction in each well and report this result without requiring a single large pixel array of a camera capable of capturing the entire plate in high-resolution. This embodiment of the sensor chip could allow for low-cost analysis of multi-well plates, single cell chambers, in the field with a small footprint cassette and not a large reader as is currently predominant. In another embodiment shown in the FIG. 27 B panel an adhesive label with a patterned sensor can be adhered to a vessel such as a bottle, and monitor the mission of light from the bottle, or light pass through the liquid contained within to monitor the reagent over time, for instance if a reagent decays turning from clear to dark, the loss in signal reported by the sensor could indicate that the reagent has expired. In yet another embodiment from FIG. 27 C, an embodiment sensor could be incorporated into a dipstick or strip which can be submerged into a vessel or reagent to again monitor the light emission from the liquid, or transmission through the liquid. Such embodiments are made possible by the use of custom sensor patterning in combination with an FPCB, DSA, or both, as well as other elements known in the art.

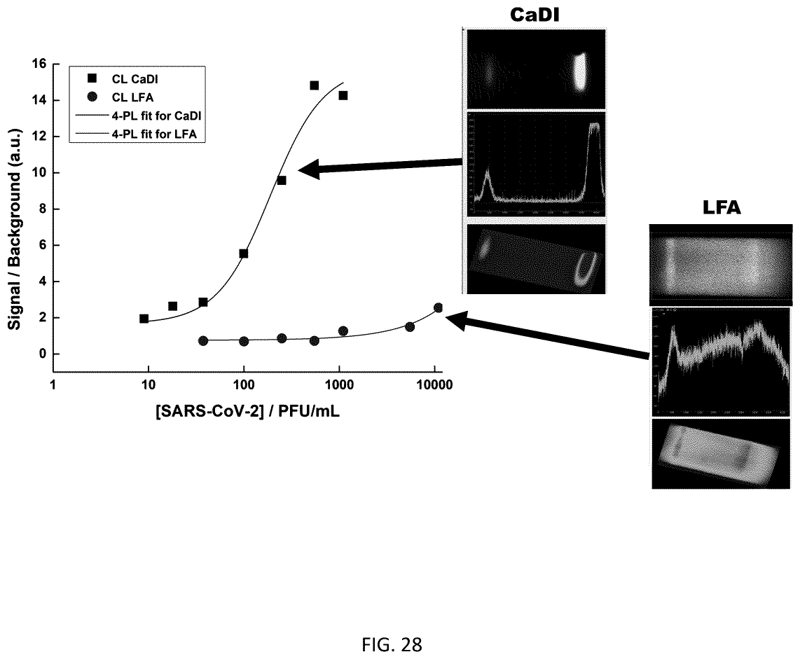

FIG. 28 shows the signal to background ratio for the CaDI system relative to a traditional Lateral Flow Assay (LFA) using chemiluminescence as the read out. The plot on the left shows the signal to noise ratio for the system while the images on the right shows actual photographs and heat maps from the two devices. Unexpectedly, the signal to noise ratio is much higher for Applicant's system than the traditional LFA. This greater sensitivity is made possible by Applicant's combination of the fluidics and the reader.

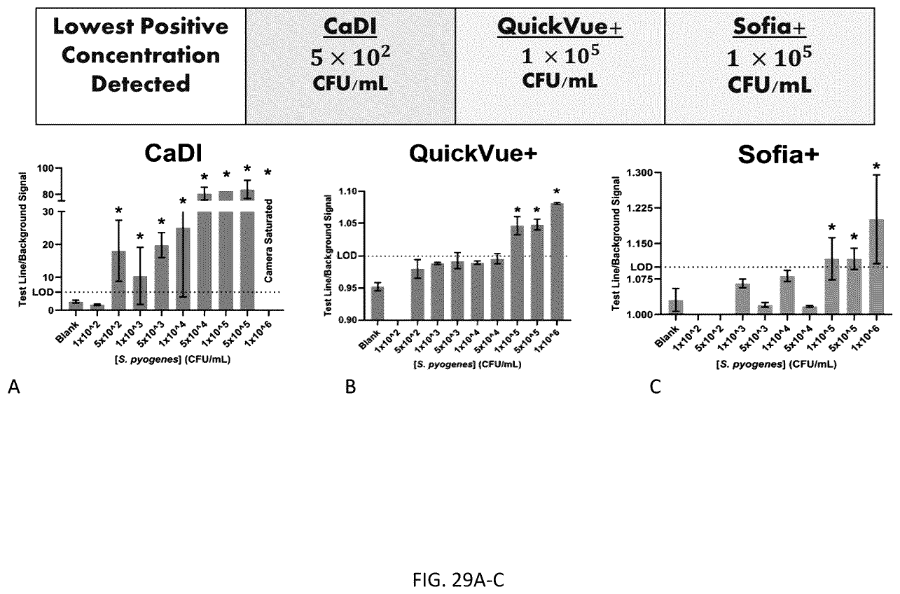

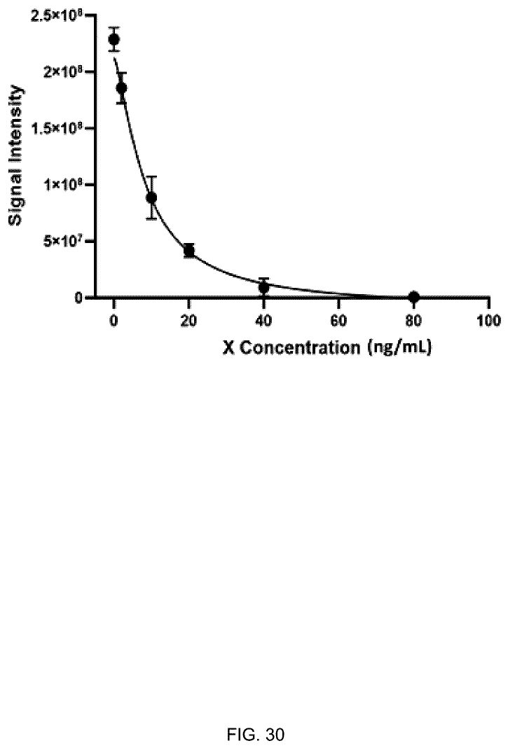

FIG. 29A-C. shows a comparison the CaDI platform (A) using chemiluminescence detection compared to two commercial products from Quidel®, the QuickVue™ (B) and the Sofia+™ (B). In these cases, Applicant used samples of Streptococcus pyogenes (S. pyogenes). The same samples were used for all measurements. Using CaDI, Applicant were able to detect 5×10² CFU/mL while both Quidel tests gave detection limits of 1×10⁵ CFU/mL. This is very surprising unexpected because it shows Applicant's CaDI platform is >100-fold more sensitive than current products on the market. This is particularly surprising for the Sofia system because it is a fluorescent immunoassay that includes a reader.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

As used herein, the term “comprise,” or variations thereof such as “comprises” or “comprising,” are to be read to indicate the inclusion of any recited integer (e.g., a feature, element, characteristic, property, method/process step or limitation) or group of integers (e.g., features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “microfluidic chip” means a device for manipulating nanoliter to microliter volumes of liquid. Such devices frequently contain features such as channels, chambers, and/or valves, and can be fabricated from a variety of different materials, including, but not limited to, glass, polyethylene terephthalate (PET), and polydimethylsiloxane (PDMS).

A “channel,” as used herein, means a feature on or in an article (substrate) that at least partially directs the flow of a fluid. The channel can have any cross-sectional shape (circular, oval, triangular, irregular, square or rectangular, or the like) and can be covered or uncovered. In embodiments where it is completely covered, at least one portion of the channel can have a cross-section that is completely enclosed, or the entire channel may be completely enclosed along its entire length with the exception of its inlet(s) and outlet(s). A channel may also have an aspect ratio (length to average cross sectional dimension) of at least 2:1, more-typically at least 3:1, 5:1, or 10:1 or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to fluid flow of less than about 5 nun or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases, the dimensions of the channel may be chosen such that fluid is able to freely flow through the article or substrate. The dimensions of the channel may also be chosen, for example, to allow a certain volumetric or linear flowrate of fluid in the channel. Of course, the number of channels and the shape of the channels can be varied by any method known to those of ordinary skill in the art. In some cases, more than one channel or capillary may be used. For example, two or more channels may be used, where they are positioned inside each other, positioned adjacent to each other, positioned to intersect with each other, etc.

“Analyte” means any chemical compound, biomolecule, bacteria, virus or portions thereof susceptible to detection and/or quantification through the device or methods of the invention. “Sample” means a composition that may or may not include an analyte.

The term “chemiluminescent” as used herein, refers to a chemical reaction which emits light as a result of a reaction without the addition of heat. In certain embodiments, a substrate of the invention may be a “chemiluminescent molecule” refers to a molecule that is capable of emitting light, often in the presence of a catalyst, in a chemiluminescent form as a result of one or more chemical processes.

“Luminol” is a chemiluminescent substrate of HRP. In the presence of peroxide, HRP oxidizes luminol to an excited product called 3-aminophthalate that emits light at 425 nm. The emission continues till 3-aminophthalate decays and enters the ground state.

As used herein, the term “sample” or “fluid” includes any fluid to be tested by the devices or methods of the invention, and may preferably include biological or environmental fluid samples, such as wastewater sample, and preferably wastewater samples containing a biological or chemical analyte. A “biological sample” includes any bodily fluid or tissue. Biological samples or samples appropriate for use according to the methods provided herein include, without limitation, blood, serum, urine, saliva, tissues, cells, and organs, or portions thereof, as well as isolated cells derived from a subject, or other organism, such as a bacterium, plant, fungi or other cell. Additional embodiment can include bone marrow, such as bone marrow aspirates, as well as cell, tissues or fluid aspirates, including fine needles aspirates. A “subject” is any organism of interest, generally a mammalian subject, and preferably a human subject.

Certain embodiments of the inventive technology may utilize a machine and/or device, such as a module, which may include a general purpose computer, a computer that can perform an algorithm, computer readable medium, software, computer readable medium continuing specific programming, a computer network, a server and receiver network, transmission elements, wireless devices and/or smart phones, internet transmission and receiving element; cloud-based storage and transmission systems, software updatable elements; computer routines and/or subroutines, computer readable memory, data storage elements, random access memory elements, and/or computer interface displays that may represent the data in a physically perceivable transformation such as visually displaying said processed data. In addition, as can be naturally appreciated, any of the steps as herein described may be accomplished in some embodiments through a variety of hardware applications including a keyboard, mouse, computer graphical interface, voice activation or input, server, receiver and any other appropriate hardware device known by those of ordinary skill in the art.

As used herein a “processor,” “processor system,” or “processing system,” which includes any suitable hardware and/or software system, mechanism or component that processes data, sensor signals or other information. A processor can include a system with a general-purpose central processing unit, multiple processing units, dedicated circuitry for achieving functionality, or other systems and for implementing one or more “computer executable program,” generally in the form of programed software-based executable instructions. Processing need not be limited to a geographic location or have temporal limitations. For example, a processor can perform its functions in “real time,” “offline,” in a “batch mode,” etc. Portions of processing can be performed at different times and at different locations, by different (or the same) processing systems. A computer may be any processor in communication with a memory. The memory may be any suitable processor-readable storage medium, such as random-access memory (RAM), read-only memory (ROM), magnetic or optical disk, or other tangible media suitable for storing instructions for execution by the processor.

Particular embodiments may be implemented by using a programmed general purpose digital computer, by using application specific integrated circuits, programmable logic devices, field programmable gate arrays, optical, chemical, biological, quantum or nano-engineered systems, components and mechanisms may be used. In general, the functions of particular embodiments can be achieved by any means as is known in the art. Distributed, networked systems, components, and/or circuits can be used. Communication, or transfer, of data may be wired, wireless, or by any other means.

It will also be appreciated that one or more of the elements depicted in the drawings/figures can also be implemented in a more separated or integrated manner, or even removed or rendered as inoperable in certain cases, as is useful in accordance with a particular application. It is also within the spirit and scope to implement a program or code that can be stored in a machine-readable medium to permit a computer to perform any of the methods described above.

For the sake of brevity, conventional techniques related to computer programming, computer networking, and other functional aspects of the systems (and the individual operating components of the systems) may not be described in detail herein. In addition, those skilled in the art will appreciate that embodiments may be practiced in conjunction with any number of system and/or network architectures, data transmission protocols, and device configurations, and that the system described herein is merely one suitable example. Furthermore, certain terminology may be used herein for the purpose of reference only, and thus is not intended to be limiting. For example, the terms “first”, “second” and other such numerical terms do not imply a sequence or order unless clearly indicated by the context.

Embodiments of the subject matter may be described herein in terms of functional and/or logical block components and with reference to symbolic representations of operations, processing tasks, and functions that may be performed by various computing components or devices. Such operations, tasks, and functions are sometimes referred to as being computer-executed, computerized, software-implemented, or computer-implemented. In this regard, it should be appreciated that the various block components shown in the figures may be realized by any number of hardware, software, and/or firmware components configured to perform the specified functions.

For example, an embodiment of a system or a component may employ various integrated circuit components, e.g., memory elements, digital signal processing elements, logic elements, look-up tables, or the like, which may carry out a variety of functions under the control of one or more microprocessors or other control devices. In this regard, the subject matter described herein can be implemented in the context of any computer-implemented system and/or in connection with two or more separate and distinct computer-implemented systems that cooperate and communicate with one another.

As used herein, the term “electrically connected” means two or more components of a system that are configured to allow the wired or wireless transmission of an electrical current.

As used herein the singular forms “a”, “an”, and “the” include plural referents unless the context clearly dictates otherwise. Thus, for example, reference to “a compound” includes a plurality of such compounds, and reference to “the method” includes reference to one or more methods, method steps, and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value.

The invention now being generally described will be more readily understood by reference to the following examples, which are included merely for the purposes of illustration of certain embodiments of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### REFERENCES

1. Ghosh, Sthitodhi, et al. “A new microchannel capillary flow assay (MCFA) platform with lyophilized chemiluminescence reagents for a smartphone-based POCT detecting malaria.” Microsystems & Nanoengineering 6.1 (2020): 1-18.
2. Shen, Minjie, et al. “An enhanced centrifugation-assisted lateral flow immunoassay for the point-of-care detection of protein biomarkers.” Lab on a Chip 20.15 (2020): 2626-2634.
3. Park, Juhwan, and Je-Kyun Park. “Pressed region integrated 3D paper-based microfluidic device that enables vertical flow multistep assays for the detection of C-reactive protein based on programmed reagent loading.” Sensors and Actuators B: Chemical 246 (2017): 1049-1055.

1. A chemiluminescent assay device comprising:
a chemiluminescent testing assembly comprising:
a sample inlet in communication with a microfluidic network formed by a first path extending to a detection area and a second path extending to the detection area, wherein said detection area is configured to capture a target analyte;
a first dried reagent disposed along the first path; and
a second dried reagent disposed along the second path,
wherein, when a fluid is provided to the sample inlet the fluid fills the microfluidic network and directs a portion of the fluid through the microfluidic channel to the detection area where a target analyte is captured, and wherein a first portion of the fluid rehydrates the first dried reagent to produce a first rehydrated reagent, a second portion of the fluid rehydrates the second dried reagent to produce a second rehydrated reagent, and the first rehydrated reagent and the second rehydrated reagent are sequentially delivered to the detection area by capillary-driven flow, and wherein the first reagent, and the second reagent react thereby emitting an chemiluminescent signal.

2. The device of claim 1, further comprising a pad, wherein the pad is disposed within the first path and contains the first dried reagent or wherein the pad is disposed within the second path and contains the second dried reagent.

3. The device of claim 1, wherein the first path includes a first surface on which the first dried reagent is disposed, or the second path includes a second surface on which the second dried reagent is disposed.

4. The device of claim 1, wherein the first rehydrated reagent arrives at the detection area before the second rehydrated reagent.

5. The device of claim 1, wherein the first path is shorter than the second path.

6. The device of claim 1, wherein the microfluidic network is configured such that the fluid arrives at the detection area by the first path before the fluid arrives at the detection area by the second path.

7-9. (canceled)

10. The device of claim 1, wherein the first rehydrated reagent comprises an enzyme label rehydrated from a dried enzyme label pad.

11. The device of claim 10, wherein the enzyme label comprises a secondary antibody directed to a target analyte.

12. The device of claim 1, wherein the second rehydrated reagent comprises a substrate rehydrated from a dried substrate pad.

13. The device of claim 12, wherein the substrate comprises a chemiluminescent substrate configured to react with the enzyme label and emit chemiluminescent signal.

14. (canceled)

15. The device of claim 1, further comprising a catalyst.

16. The device claim 15, wherein the catalyst is disposed of within the fluid, or within a buffer, or on the dried substrate pad, or on a test strip.

17. The device claim 16, wherein the fluid comprises a biological sample, an environmental sample, a food sample, or a water sample.

18-19. (canceled)

20. The device of claim 1, wherein said detection area comprises a test strip in communication with said microfluidic network.

21. The device of claim 20, wherein said test strip comprise a capture probe configured to bind to a target analyte.

22. The device of claim 21, wherein said test strip is in communication with a passive pump configured to generate capillary-driven flow through said microfluidic network.

23. (canceled)

24. The device of claim 1, wherein said fluid washes the detection area prior to the first rehydrated reagent contacts the detection area.

25-50. (canceled)

51. An apparatus for analyzing a chemiluminescent assay, the apparatus comprising:
a chemiluminescent assay device;
a sensor; and
a control system, wherein:
the sensor is configured to receive a chemiluminescent response from the chemiluminescent assay device; and
the control system is configured to provide feedback to a user.

52-72. (canceled)

73. An apparatus for analyzing a chemiluminescent assay, the apparatus comprising:
a chemiluminescent assay device;
a sensor;
a lens system; and
a control system, wherein:
the sensor is configured to receive a chemiluminescent response from the chemiluminescent assay device and lens system;
the control system is configured to provide feedback to a user; and
the chemiluminescent assay device comprises a microfluidic network comprising a delay element.

74-93. (canceled)

94. The device of claim 1, further comprising:
a sensor; and
a control system, wherein:
the sensor is configured to receive a chemiluminescent response from the detection area; and
the control system is configured to provide feedback to a user.

95. The device of claim 94, further comprising a light guide, wherein the light guide is configured to direct the chemiluminescent response from the chemiluminescent assay device to the sensor.

96. The device of claim 94, wherein the sensor is selected from a complementary metal-oxide-semiconductor (CMOS) sensor, a back-illuminated sensor, a back-thinned sensor, a monochrome sensor, or a stacked circuit with a pixel micro lens array.

97. The device of claim 95, wherein the light guide comprises a fiber optic plate.

98. The device of claim 97, wherein the fiber optic plate is bonded to the chemiluminescent assay device, or to the sensor.

99. The device of claim 94, further comprising an activation switch that is an open electrical circuit which is closed upon introduction of a liquid sample to the apparatus.

100. The device of claim 1, further comprising:
a sensor;
a light guide; and
a control system, wherein:
the sensor is configured to receive a chemiluminescent response from the detection area and light guide;
the control system is configured to provide feedback to a user; and
the chemiluminescent assay device comprises a microfluidic network comprising a delay element.

101. The device of claim 100, wherein the delay element comprises a convoluted portion of microfluidic network, or a portion of the microfluidic network channels with an increased cross-sectional area.

102. The device of claim 100, wherein the microfluidic network comprises a centering element configured to direct flow through the microfluidic network to the center of a test strip.

103. The device of claim 102, wherein the microfluidic network is in fluid communication with an enzyme pad and a chemiluminescent substrate pad.

104. The device of claim 103, wherein the delay element is configured to provide for a flow time through the microfluidic network from the chemiluminescent substrate pad to the test strip that is greater than the flow time from the enzyme pad to the test strip.
